(19) United States
(12) Patent Application Publication
(10) Pub. No.: US 2023/0067496 A1
(43) Pub. Date: Mar. 2, 2023

(54) TREATMENT OF OTIC DISEASES AND CONDITIONS

(71) Applicant: Spiral Therapeutics Inc., South San Francisco, CA (US)

(72) Inventors: Eugene de Juan, Jr., San Francisco, CA (US); Andrew Ayoob, San Francisco, CA (US); Signe Erickson, San Francisco, CA (US); Kathleen Farinas, Los Altos, CA (US)

(21) Appl. No.: 17/797,606

(22) PCT Filed: Feb. 12, 2021
(86) PCT No.: PCT/US2021/018019

§ 371 Date: Aug. 4, 2022

Related U.S. Application Data
(60) Provisional application No. 62/976,030, filed on Feb. 13, 2020.

(51) Int. Cl. A61K 9/06; C07K 16/22; A61K 45/06; A61K 9/00

(52) U.S. Cl. CPC A61K 9/06; C07K 16/22; A61K 45/06; A61K 9/0046

(57) ABSTRACT

Active agents that bind to VEGF or a VEGF receptor and reduce the severity of a condition associated with BLB disruption and/or angiogenesis, for example anti-VEGF antibodies or tyrosine kinase inhibitor small molecules, can be locally, regionally or systemically administered to an individual with an otic disease or condition, such as Méniére's Disease, Autoimmune Inner Ear Disease, sensorineural hearing loss and noise-induced hearing loss, to alleviate symptoms of the disease or condition, for example, due to edema and endolymphatic dysfunction. An effective amount of these compounds can be delivered by intratympanic or intracochlear administration. Other methods of administration include, but are not limited to, topical, parenteral, subcutaneous, intraperitoneal and intranasal. Formulations may be, for example, for immediate release, sustained release, or controlled release.

### CROSS-REFERENCE TO RELATED APPLICATIONS

This application claims priority to U.S. Provisional Application Serial No. 62/976,030, filed on Feb. 13, 2020, the entire contents of which are incorporated by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: 50051_0010WO1.txt, date recorded, Feb. 12, 2021, file size≈47 kilobytes.

### TECHNICAL FIELD

The present disclosure relates to treatment of vascular and physiological dysfunction associated with otic disorders and conditions, such as Méniére's Disease (MD), sensorineural hearing loss, age-related hearing loss, noise-induced hearing loss, autoimmune ear disease, tinnitus, and other ear disorders. In particular, the disclosure relates to targeting the involvement of the vascular endothelial growth factor (“VEGF”) pathway and homologues involved in the etiology of otic disorders and conditions.

### BACKGROUND

Some otic disorders or conditions have underlying microvascular etiology. Non-limiting examples of such disorders or conditions include Méniére's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, and similar ear disorders and conditions. Typically, such disorders have no treatment, other than for some of the symptoms, nor a cure.

Méniére's Disease is a chronic, incurable inner ear disorder with recurrent debilitating symptoms that affect hearing and balance. It is named for French physician Prosper Ménière who, in 1861, first identified and described the symptoms of this medical condition.

Researchers are unsure of what causes the buildup of fluid in the inner ear that results in MD. Some believe it is related to vascular insufficiencies, others say it might be due to autoimmune conditions, viral infections, allergic reactions or that the disease may initiate from a trauma. In some cases, MD appears to have a hereditary component, so a gene mutation may be connected to the regulation of inner ear fluid.

Autoimmune Inner Ear Disease is a rare disorder, appearing in both adults and children, caused by an immune system response. The inner ear can be the direct target of the immune response, but it can be additionally damaged by a deposition of circulating immune complexes or by systemic immune-mediated diseases. The clinical expression of immune-mediated inner ear disease can show a progressive bilateral and asymmetric sensorineural hearing loss (SNHL) profile. Cochlear symptoms are often associated with vestibular disorders. In about 50% of AIED patients, hearing loss is also associated with vestibular symptoms, such as imbalance and motion intolerance, ataxia and positional or episodic vertigo.

Sensorineural Hearing Loss is due to impaired ability of the cochlea to effectively transduce pressure waves into neural signaling. SNHL is typically associated with exposure to loud noise, aging, head trauma, exposure to ototoxic drugs, infection, autoimmune disease, Méniére's disease, genetic mutations, and tumors of the auditory nerve.

Noise-induced hearing loss is caused by exposure to loud and/or long-lasting sounds. Hearing loss-may occur from prolonged exposure to loud noises, such as heavy machinery, loud music, airplanes or gunfire. Long, repeated or impulse exposure to sounds at or above 85 decibels can cause hearing loss. NIHL causes damage to the hair cells and/or the auditory nerve.

Symptoms of MD, AIED, SNHL, NIHL, and other ear disorders can include vertigo, hearing loss, ear ringing (tinnitus), and/or ear pressure. In some cases, the vertigo may cause severe nausea and imbalance. Hearing loss may become permanent. There is no treatment, other than for some of the symptoms, nor a cure. Drugs for motion sickness or nausea may help manage the symptoms.

It is therefore an object of the present disclosure to provide treatments for otic diseases and conditions, such as MD, AIED, SNHL (e.g., SSNHL, sensorineural hearing loss associated with diabetes), NIHL, age-related hearing loss, tinnitus, and similar ear disorders and conditions.

### SUMMARY

The blood labyrinthine barrier (BLB) is important in the maintenance of inner ear ionic and fluid homeostasis. Imaging and histopathology demonstrate loss of integrity of the BLB in the affected inner ear of patients suffering from otic disorders and conditions, including Méniére's disease (MD); see, e.g., FIG. 1. Environmental inflammatory stimuli, acoustic trauma, autoimmune disorders, transient hypoxia, or other insults can contribute to a loss of vascular homeostasis in the inner ear. This causes an inflammatory stimulus that triggers compensatory up-regulation of vascular endothelial growth factor (VEGF), leading to disruption of the angiogenesis of the BLB, especially in the macula utricle. While these disorders and conditions may vary in underlying causality, a common unifying factor is subsequent disruption of the inner ear microvasculature and/or BLB. Without being bound by any particular theory, it is believed that inhibiting VEGF signaling in the inner ear, microvascular integrity can be restored, and effectively prevent further progression of hearing loss and vestibular symptoms, independent of original causality.

Active agents which bind to VEGF or a VEGF receptor (VEGFR) and that may reduce the severity of conditions associated with a BLB vascular and/or lymphatic pathology include, for example, VEGF inhibitors such as antibodies or tyrosine kinase inhibitor small molecules that block signaling through one or more VEGFRs. These therapeutic compounds can be locally, regionally or systemically administered to an individual with an otic disease or condition (e.g., (MD, AIED, SNHL (e.g., SSNHL or sensorineural hearing loss associated with diabetes), NIHL, age-related hearing loss, tinnitus, or a combination thereof) to alleviate symptoms of the disease or condition by reducing edema and/or lymphatic dysfunction.

An effective amount of these compounds are preferably delivered by transtympanic or intracochlear administration. Other methods of administration include, but are not limited to, parenteral, subcutaneous, intraperitoneal, intranasal, intravenous and oral. Formulations may be for immediate release, sustained or controlled release. Dosage forms may include solutions, suspensions, ointments, hydrogels, liposomes, controlled release particles, tablets and capsules. Delivery of an active agent may also be via an implantable device. Other compounds that both reduce edema and inhibit lymphatic dysfunction (e.g., leaky junction associated with MD), can be used similarly to VEGF inhibitors. Steroids are not typically used as part of the treatment, nor are the VEGF inhibitors typically used in the same dosage as used in the treatment of cancer. The VEGF inhibitor may, in some cases, be administered in combination with one or more other therapeutic agents. In some aspects, provided herein is a method of treating an otic disease or condition in a subject, the method including (i) identifying a subject as having an otic disease or condition, and (ii) administering a therapeutically effective dose of a VEGF inhibitor to the subject. In some aspects, provided herein is a method of treating an otic disease or condition in a subject, the method including administering a therapeutically effective dose of a VEGF inhibitor to a subject in need thereof.

In some embodiments, the administering includes systemic administration. In some embodiments, the administering includes administering to an affected ear of the subject. In some embodiments, the VEGF inhibitor is administered in suspension, an ointment, a hydrogel, a liposome, a controlled release particle, an implantable device, or a combination thereof. In some embodiments, the VEGF inhibitor is administered as in a formulation.

In some aspects, also provided herein is a method of treating an otic disease or condition in a subject, the method including (i) preparing a formulation including a VEGF inhibitor, and (ii) administering a therapeutically effective dose of the formulation to an affected ear of a subject in need thereof. In some aspects, provided herein is a method of treating an otic disease or condition in a subject, the method including (i) identifying a subject as having an otic disease or condition, (ii) preparing a formulation including a VEGF inhibitor, and (iii) administering a therapeutically effective dose of the formulation to an affected ear of the subject.

In some embodiments, identifying a subject as having an otic disease or condition includes identifying one or more symptoms of an otic disease or condition in the subject. In some embodiments, identifying a subject as having an otic disease or condition includes identifying presence of the blood labyrinthine barrier (BLB) leakage. In some embodiments, following identifying the subject as having an otic disease or condition, the method further includes identifying presence of BLB leakage.

In some embodiments, the administering includes administering between about 5 μL and about 500 μL of the formulation. In some embodiments, the administering includes administering between about 25 μL and about 200 μL of the formulation.

In some embodiments, the formulation includes a hydrogel forming agent. In some embodiments, the formulation can form a hydrogel in the affected ear of the subject. In some embodiments, the hydrogel is selected from the group consisting of a temperature mediated phase transition gel, a shear thinning gel, an ionically crosslinked gel, a dendrimer gel, and a combination thereof. In some embodiments, the hydrogel is a temperature mediated phase transition gel. In some embodiments, the hydrogel forming agent is a poloxamer. In some embodiments, the hydrogel forming agent is poloxamer 407. In some embodiments, the hydrogel is a shear thinning gel. In some embodiments, the hydrogel is an ionically crosslinked gel. In some embodiments, the hydrogel forming agent is alginate. In some embodiments, the hydrogel is a dendrimer gel. In some embodiments, the hydrogel forming agent is present in the formulation in an amount of about 5% (w/w) to 30% (w/w). In some embodiments, the hydrogel forming agent is present in the formulation in an amount of about 15% (w/w) to 30% (w/w). In some embodiments, the hydrogel forming agent is present in the formulation in an amount of about 20% (w/w) to 30% (w/w).

In some embodiments, the VEGF inhibitor is present in the formulation in an amount of about 0.003% (w/w) to about 20% (w/w). In some embodiments, the VEGF inhibitor is present in the formulation in an amount of about 0.01% (w/w) to about 20% (w/w). In some embodiments, the VEGF inhibitor is present in the formulation in an amount of about 0.5% (w/w) to about 20% (w/w).

In some embodiments, the formulation further includes one or more of an antimicrobial, an antioxidant, a buffer, a carrier, a chelator, a crystallization inhibitor, a detergent, a mucoadhesive agent, a penetration enhancer, a preservative, a solubilizing agent, a stabilizer, a tonicity agent, or a viscosity modifier. In some embodiments, the formulation further includes a steroid, a diuretic, a vasodilator, an antiinfective, an antihistamine, or a combination thereof.

In some embodiments, the formulation, following administration, provides sustained release of the VEGF inhibitor for at least 3 days. In some embodiments, the formulation, following administration, provides sustained release of the VEGF inhibitor for at least 1 week. In some embodiments, the formulation, following administration, provides sustained release of the VEGF inhibitor for at least 3 weeks. In some embodiments, the formulation, following administration, provides sustained release of the VEGF inhibitor for at least 1 month. In some embodiments, the formulation, following administration, provides sustained release of the VEGF inhibitor for at least 3 months.

In some embodiments, the administering of the VEGF inhibitor includes injecting through the tympanic membrane. In some embodiments, the administering of the VEGF inhibitor includes administering to the cochlea. In some embodiments, the administering of the VEGF inhibitor includes administration to the tympanic cavity. In some embodiments, the administering includes application of the VEGF inhibitor on the tympanic membrane. In some embodiments, the administering includes application of the VEGF inhibitor into the cochlea by injection, direct instillation or perfusion of the inner ear compartments. In some embodiments, the administering occurs during a surgical procedure selected from the group consisting of cochleostomy, labyrinthotomy, mastoidectomy, stapedectomy, endolymphatic sacculotomy, and a combination thereof.

In some embodiments, VEGF inhibitor is selected from the group consisting of altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof. In some embodiments, the VEGF inhibitor includes an antibody or antigen-binding fragment thereof. In some embodiments, the antibody or antigen-binding fragment thereof is selected from the group consisting of alacizumab, bevacizumab, icrucumab, ramucirumab, ranibizumab, and combinations thereof.

In some embodiments, the VEGF inhibitor includes a decoy receptor. In some embodiments, the decoy receptor is aflibercept. In some embodiments, the VEGF inhibitor includes an allosteric modulator of a VEGFR. In some embodiments, the allosteric modulator of a VEGFR is cyclotraxin B. In some embodiments, the VEGF inhibitor is at least 10-fold selective for VEGFR2 over another VEGFR. In some embodiments, the VEGF inhibitor is at least 20-fold selective for VEGFR2 over another VEGFR. In some embodiments, the VEGF inhibitor is at least 50-fold selective for VEGFR2 over another VEGFR. In some embodiments, the amount of the VEGF inhibitor is sufficient to reduce edema and/or lymphatic dysfunction in an affected ear.

In some embodiments, the otic disease or condition can be selected from the group consisting of Méniére's Disease (MD), Autoimmune Inner Ear Disease (AIED), sensorineural hearing loss (SNHL), noise-induced hearing loss (NIHL), age-related hearing loss, tinnitus, and a combination thereof. In some embodiments, the otic disease or condition can be selected from the group consisting of Autoimmune Inner Ear Disease (AIED), sensorineural hearing loss (SNHL), noise-induced hearing loss (NIHL), age-related hearing loss, tinnitus, and a combination thereof. In some embodiments, the SNHL can be selected from the group consisting of sudden sensorineural hearing loss, sensorineural hearing loss associated with diabetes, and a combination thereof. In some embodiments, the otic disease or condition is not Méniére's Disease.

In some aspects, also provided herein is use of a VEGF inhibitor for the treatment of an otic disease or condition. In some aspects, also provided herein is use of a VEGF inhibitor in the manufacture of a medicament for the treatment of an otic disease or condition.

In some embodiments, the VEGF inhibitor is selected from the group consisting of altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof. In some embodiments, the VEGF inhibitor includes an antibody or antigen-binding fragment thereof. In some embodiments, the antibody or antigen-binding fragment thereof is selected from the group consisting of alacizumab, bevacizumab, icrucumab, ramucirumab, ranibizumab, and combinations thereof.

In some embodiments, the VEGF inhibitor includes a decoy receptor. In some embodiments, the decoy receptor is aflibercept. In some embodiments, the VEGF inhibitor includes an allosteric modulator of a VEGFR. In some embodiments, the allosteric modulator of a VEGFR is cyclotraxin B. In some embodiments, the VEGF inhibitor is at least 10-fold selective for VEGFR2 over another VEGFR. In some embodiments, the VEGF inhibitor is at least 20-fold selective for VEGFR2 over another VEGFR. In some embodiments, the VEGF inhibitor is at least 50-fold selective for VEGFR2 over another VEGFR. In some embodiments, the amount of the VEGF inhibitor is sufficient to reduce edema and/or lymphatic dysfunction in an affected ear.

In some embodiments, the VEGF inhibitor is in the form of a suspension, an ointment, a hydrogels, a liposome, a controlled release particle, an implantable device, or a combination thereof.

In some embodiments, the VEGF inhibitor is part of a formulation. In some embodiments, an effective dose of the formulation is about 5 μL to about 500 μL of the formulation. In some embodiments, an effective dose of the formulation is about 50 μL to about 200 μL of the formulation. In some embodiments, the formulation includes a hydrogel forming agent. In some embodiments, formulation can form a hydrogel in the affected ear of the subject. In some embodiments, the hydrogel is selected from the group consisting of a temperature mediated phase transition gel, a shear thinning gel, an ionically crosslinked gel, a dendrimer gel, and a combination thereof. In some embodiments, the hydrogel is a temperature mediated phase transition gel. In some embodiments, the hydrogel forming agent is a poloxamer. In some embodiments, the hydrogel forming agent is poloxamer 407. In some embodiments, the hydrogel is a shear thinning gel. In some embodiments, the hydrogel is an ionically crosslinked gel. In some embodiments, the hydrogel forming agent is alginate. In some embodiments, the hydrogel is a dendrimer gel. In some embodiments, the hydrogel forming agent is present in the formulation in an amount of about 5% (w/w) to 30% (w/w). In some embodiments, the hydrogel forming agent is present in the formulation in an amount of about 15% (w/w) to 30% (w/w). In some embodiments, the hydrogel forming agent is present in the formulation in an amount of about 20% (w/w) to 30% (w/w). In some embodiments, the VEGF inhibitor is present in the formulation in an amount of about 0.003% (w/w) to about 20% (w/w). In some embodiments, the VEGF inhibitor is present in the formulation in an amount of about 0.01% (w/w) to about 10% (w/w). In some embodiments, the VEGF inhibitor is present in the formulation in an amount of about 0.5% (w/w) to about 10% (w/w). In some embodiments, the formulation further includes one or more of an antimicrobial, an antioxidant, a buffer, a carrier, a chelator, a crystallization inhibitor, a detergent, a mucoadhesive agent, a penetration enhancer, a preservative, a solubilizing agent, a stabilizer, a tonicity agent, or a viscosity modifier. In some embodiments, the formulation further includes a steroid, a diuretic, a vasodilator, an antiinfective, an antihistamine, or a combination thereof. In some embodiments, the formulation, following administration, can provide sustained release of the VEGF inhibitor for at least 3 days. In some embodiments, the formulation, following administration, can provide sustained release of the VEGF inhibitor for at least 1 week. In some embodiments, the formulation, following administration, can provide sustained release of the VEGF inhibitor for at least 3 weeks. In some embodiments, the formulation, following administration, can provide sustained release of the VEGF inhibitor for at least 1 month. In some embodiments, the formulation, following administration, can provide sustained release of the VEGF inhibitor for at least 3 months. Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

In some embodiments, the otic disease or condition can be selected from the group consisting of Méniére's Disease (MD), Autoimmune Inner Ear Disease (AIED), sensorineural hearing loss (SNHL), noise-induced hearing loss (NIHL), age-related hearing loss, tinnitus, and a combination thereof. In some embodiments, the otic disease or condition can be selected from the group consisting of Autoimmune Inner Ear Disease (AIED), sensorineural hearing loss (SNHL), noise-induced hearing loss (NIHL), age-related hearing loss, tinnitus, and a combination thereof. In some embodiments, the SNHL can be selected from the group consisting of sudden sensorineural hearing loss, sensorineural hearing loss associated with diabetes, and a combination thereof. In some embodiments, the otic disease or condition is not Méniére's Disease.

The details of one or more embodiments of the disclosure are set forth in the accompanying drawings and the description below. Other features, objects, and advantages of the disclosure will be apparent from the description and drawings, and from the claims. The word “comprising” in the claims may be replaced by “consisting essentially of” or with “consisting of,” according to standard practice in patent law.

### BRIEF DESCRIPTION OF THE DRAWINGS

FIG. 1 shows a schematic of some of the effects of Méniére's Disease in the ear.

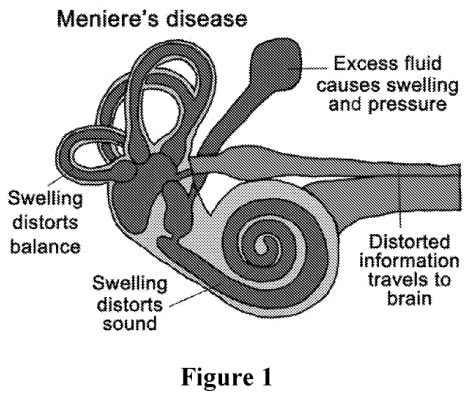

### DETAILED DESCRIPTION

I. Definitions

“Active agent” and “active pharmaceutical ingredient” are used interchangeably and refer to a physiologically or pharmacologically active substance that acts locally and/or systemically in the body. An active agent is a substance that is administered to a patient for the treatment, prevention, or diagnosis of a disease or disorder.

The term “ADME” is an abbreviation in pharmacokinetics and pharmacology for “absorption, distribution, metabolism, and excretion”, and describes the disposition of an active agent (e.g., a drug) within an organism. These four criteria all influence the drug levels and kinetics of drug exposure to the tissues and hence influence the performance and pharmacological activity of the compound as a drug.

The term “AUC” or “area under the curve” in the field of pharmacokinetics, is the definite integral in a plot of an active agent (e.g., drug) concentration in blood plasma versus time. In practice, the active agent (e.g., drug) concentration is typically measured at certain discrete points in time and the trapezoidal rule is used to estimate AUC. The AUC of an active agent is typically used to evaluate the exposure of a subject to an active agent over time.

The term “blood labyrinth barrier” or “BLB” refers to the barrier between the vasculature and the inner ear fluids, either endolymph or perilymph. The BLB is involved in the maintenance of the inner ear fluid ionic homeostasis.

The term “BLLQ” is an abbreviation for “below the lower limit of quantification” and is defined as below the lowest standard on a calibration curve.

The term “Cₘₐₓ” refers to the maximum (or peak) serum concentration that an active agent (e.g., a drug) achieves (e.g., systemically or in a specified compartment or test area of the subject) after the active agent (e.g., drug) has been administered. In some embodiments, Cₘₐₓ is measured before the administration of a second dose of the active agent.

The term “Cₘᵢₙ”n refers to the minimum (or trough) serum concentration that an active agent (e.g., a drug) achieves after dosing (e.g., systemically, or in a specified compartment or test area of the subject) after the active agent has been administered. In some embodiments, Cₘᵢₙ is measured before the administration of a second dose of the active agent).

The term “degree of functionalization” when referring to crosslinking, is the number of functional groups per appropriate polymeric unit (e.g., polymer chain, branch, or monomer) that are suitable for crosslinking using a given crosslinker. For example, if a polymer has one functional group per monomer, then the appropriate polymeric unit is a monomer. As another example, if a polymer has one functional group per branch terminus, then the appropriate polymeric group is a branch.

The term “co-administration”, as used herein, are meant to encompass, generally, administration of two or more active agents to a single subject, and are intended to include prevention regimens in which the active agents are administered by the same or different route of administration or at the same or different time.

The term “drug absorption” or “absorption” refers, typically, to the process of movement of the active agent from the localized site of administration to a site of therapeutic effect. In some cases, drug absorption can be through the round window niche of the cochlea, and across a barrier (e.g., the round window membrane) into one or more inner ear structures.

The abbreviation “DDI” refers to drug-drug interaction.

The phrase “effective amount” or “effective concentration” means an amount of active agent that, when at a site of action, is sufficient to (i) treat a disease or disorder, (ii) attenuate, ameliorate, or eliminate one or more symptoms of the particular disease, condition, or disorder, or (iii) delay the onset of one or more symptoms of the particular disease, condition, or disorder described herein. The amount of an active agent that will correspond to such an amount will vary depending upon factors such as the particular active agent, disease condition and its severity, the identity (e.g., age and/or weight) of the patient in need of treatment, but can nevertheless be routinely determined by one skilled in the art. The terms “effective amount” or “therapeutically effective amount,” as used herein, can refer to a sufficient amount of an active agent at a site of action that would be expected to relieve to some extent one or more of the symptoms of the disease or condition being treated. In some embodiments, an effective amount of an active agent is a quantity necessary to render a desired anti-inflammatory result at a site of action. The term “therapeutically effective amount” includes, for example, an “effective amount” of an active agent to achieve a desired pharmacologic effect without undue adverse side effects.

The phrase “effective dose” means an amount of active agent that, when administered to a patient in need of such treatment, is sufficient to (i) treat a disease or disorder, (ii) attenuate, ameliorate, or eliminate one or more symptoms of the particular disease, condition, or disorder, or (iii) delay the onset of one or more symptoms of the particular disease, condition, or disorder described herein. In some embodiments, an “effective dose” is an amount of active agent, when administered to a patient in need of such treatment, achieves a sufficient concentration at a site of action to (i) treat a disease or disorder, (ii) attenuate, ameliorate, or eliminate one or more symptoms of the particular disease, condition, or disorder, or (iii) delay the onset of one or more symptoms of the particular disease, condition, or disorder described herein for a period of time. The dose of an active agent that will correspond to such an amount will vary depending upon factors such as the particular active agent, disease condition and its severity, the identity (e.g., age and/or weight) of the patient in need of treatment, but can nevertheless be routinely determined by one skilled in the art. The terms “effective dose” or “therapeutically effective dose,” as used herein, can refer to a sufficient amount of an active agent being administered that would be expected to relieve to some extent one or more of the symptoms of the disease or condition being treated. In some embodiments, an effective dose of an active agent is a quantity necessary to render a desired anti-inflammatory result. The term “therapeutically effective dose” includes, for example, an “effective dose” of an active agent to achieve a desired pharmacologic effect without undue adverse side effects. It will also be understood that “an effective dose” in an extended-release dosing format may differ from “an effective dose” in an immediate-release dosing format based upon pharmacokinetic and/or pharmacodynamic considerations.

The term “enhance” or “enhancing,” can refer to an increase in potency or a prolongation of a desired effect. In some cases, “enhance” or “enhancing” can also refer to a decrease of one or more adverse effects associated with an active agent. For example, in reference to enhancing the effect of the active agents disclosed herein, the term “enhancing” can refer to the ability to increase the potency or prolong the duration of effect of the active agent by an anti-inflammatory agent. An “enhancing-effective amount,” as used herein, refers to an amount of an agent that is adequate to enhance the effect of an active agent in a desired system. The amount of an agent that will correspond to such an amount will vary depending upon factors such as the particular active agent, disease condition and its severity, the identity (e.g., age and/or weight) of the patient in need of treatment, but can nevertheless be routinely determined by one skilled in the art.

The term “GLP” refers to “good laboratory practice” and is a set of principles intended to assure the quality and integrity of non-clinical laboratory studies.

The term “IC₅₀” refers to the concentration of an inhibitor at which an assayed outcome is reduced by 50%.

The term “inhibit” can mean to reduce or decrease an activity (e.g., signaling activity) or expression (e.g., of a gene). The term can also include preventing, slowing, or reversing the development of a disease or condition or the advancement of a disease or condition in a necessitating prevention. This can be a complete subject. In some cases, inhibition can be partial. In some cases, inhibition can be complete. In some embodiments, a level of inhibition can be determined based on comparison to a control or to a standard level.

The term “macromolecule” generally refers to a molecule that greater than 1500 g/mol, greater than about 2000 g/mol, or greater than about 2500 g/mol. In some forms, a macromolecule can be polymeric and/or oligomeric.

The term “MRSD” or “maximum recommended starting dose” refers to the highest amount of an agent that can be given safely and without complication while maintaining its efficacy.

The term “MTD” or “maximum tolerated dose” refers to the highest dose of a drug or prevention that does not cause unacceptable side effects.

The term “NOAEL” refers to “no observed adverse effect level” and is an important part of the non-clinical risk assessment.

The terms “otic” and “auris” refer to relating to the ear. For example, an otic composition can be a composition intended for administration to the ear.

The term “pharmaceutically acceptable” indicates that the compound, or salt or composition thereof is compatible chemically and/or toxicologically with the other ingredients comprising a formulation and/or the patient being treated therewith. In some embodiments, a pharmaceutically acceptable salt can be a salt that conserves the efficiency and/or the biological properties of the free bases or free acids. In some embodiments, a pharmaceutically acceptable salt can be a salt that change the efficiency and/or the biological properties of the free bases or free acids; for example, a pharmaceutically acceptable salt can improve the bioavailability of a free base or free acid.

The term “pharmaceutical combination”, as used herein, refers to a pharmaceutical therapy resulting from the mixing or combining of more than one active agent and includes both fixed and non-fixed combinations of the active agents. The term “fixed combination” means that a first active agent or a pharmaceutically acceptable salt or solvate thereof and at least one additional active agent, are both administered to a patient simultaneously in the form of a single composition or dosage. The term “non-fixed combination” means that a first active agent or a pharmaceutically acceptable salt or solvate thereof and at least one additional active agent are formulated as separate compositions or dosages, such that they may be administered to a subject in need thereof simultaneously, concurrently or sequentially with variable intervening time limits, using the same or different routes of administration, wherein such administration provides effective levels of the two or more compounds in the body of the subject. In one embodiment, the first active agent and the second active agent are formulated as separate unit dosage forms, wherein the separate dosage forms are suitable for either sequential or simultaneous administration. These also apply to cocktail therapies, e.g., the administration of three or more active ingredients.

The term “VEGF inhibitor” includes any agent (e.g., a small molecule, antibody, or antigen-binding fragment thereof) exhibiting inhibition of vascular endothelial growth factor (VEGF) signaling. In some embodiments, a VEGF inhibitor can bind to a vascular endothelial growth factor receptor (a VEGFR). In some embodiments, a VEGF inhibitor can bind to a vascular endothelial growth factor (e.g., a ligand of a VEGFR).

The term “auris-acceptable penetration enhancer” or “penetration enhancer” refers to an agent that reduces barrier resistance (e.g., barrier resistance of the round window membrane).

The term “pharmacodynamic” refers to the factors that determine the biologic response observed relative to the concentration of drug at the desired site, such as within the auris media and/or auris interna.

The term “pharmacokinetics” refers to factors that determine the attainment and maintenance of the appropriate concentration of drug at the desired site, such as within the auris media and/or auris interna.

The term “prophylactically effective amount” or “prophylactically effective dose” means an amount of active agent that, when administered to a patient in need of such treatment, is sufficient to (i) prevent a disease or disorder, (ii) attenuate, ameliorate, or eliminate one or more symptoms of the particular disease, condition, or disorder, before it occurs. In some cases, a “prophylactically effective amount” refers to an amount of a composition administered to a subject susceptible to or otherwise at risk of a particular disease, disorder or condition, for example, a prophylactically effective amount of an active agent can be an amount effective to prevent or to attenuate ototoxicity. For example, an apoptotic inhibitory formulation may be administered to an individual prior to chemotherapy to prevent hearing loss by a subsequently administered chemotherapeutic agent.

The term “residence time” as used herein can refer to the amount of time that a formulation remains in the location of administration. In some embodiments, residence time can be the time when there is no gel visualized on the round window membrane area, e.g., after collecting the gel at a time after injection.

The term “room temperature” refers to a temperature between about 15° C. and less than about 27° C., preferably about 25° C. or more preferably about 20° C. The term “body temperature” refers to a temperature between about 36.5° C. and about 37.5° C., preferably about 37° C.

The term “ROS” or “reactive oxygen species” are chemically reactive chemical species containing oxygen.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented.

“Small molecule” generally refers to a molecule that is less than about 1500 g/mol, less than about 1000 g/mol, less than about 800 g/mol, or less than about 500 g/mol. In some forms, small molecules are non-polymeric and/or non-oligomeric. In some embodiments, a small molecule can be organic. In some embodiments, a small molecule can be inorganic. In some embodiments, a small molecule can include both organic and inorganic atoms.

“Steady state,” can refer to when the amount of drug administered (e.g., auris media and/or auris interna) is equal to the amount of drug eliminated within one dosing interval resulting in a plateau or constant levels of drug exposure within the targeted structure.

“Stable” as used herein can refer to chemical and/or physical stability over a time period under defined conditions. In some embodiments, a stable solution can retain a high percentage or all of what was originally dissolved remaining in solution. In some embodiments, a solution can retain more than 60, 70, 80, 90, 95, 98, 99, or 100% of the originally dissolved solute at room temperature (approximately 15-25° C., most preferably 25° C.).

“Sustained release” as used herein refers to release of a substance over an extended period of time. In some embodiments, this can be contrasted with to a bolus type administration in which the entire amount of the substance is made biologically available at one time.

The term “Tₘₐₓ” refers to the time it takes a drug or other substance to reach the maximum concentration Cₘₐₓ.

The term “transtympanic administration” refers to the administration of an active agent (e.g., a therapeutic agent) via the tympanic cavity, preferably via a needle that accesses the tympanic cavity (middle ear) by penetrating the tympanic membrane (eardrum).

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (e.g., not worsening) state of disease, delay or slowing of disease progression, amelioration or palliation of the disease state (e.g., one or more symptoms of the disease), and remission (whether partial or total), whether detectable or undetectable.

The term “unit dosage form” refers to physically discrete units suitable as unitary dosages for human subjects and other patients, each unit containing a predetermined quantity of active material (e.g., an active agent as provided herein) calculated to produce the desired therapeutic effect, in association with a suitable pharmaceutical excipient.

The terms “prevent,” “preventing” or “prevention,” as used herein means the prevention of the onset, recurrence or spread, in whole or in part, of a disease or condition as described herein, or a symptom thereof.

II. Therapeutic and/or Prophylactic Formulations

A. Active Agents for Treatment of Otic Diseases and Conditions

Active agents (e.g., therapeutic compounds) that bind to VEGF or a VEGF receptor (VEGFR) and reduce the severity of a condition (e.g., MD, or any of the otic diseases or conditions described herein) associated with a BLB vascular and/or lymphatic pathology, such as VEGF inhibitors, include, for example, anti-VEGF antibodies or tyrosine kinase inhibitor small molecules. These active agents can be locally, regionally, or systemically administered to an individual with an otic disease or condition (e.g., MD, AIED, or any of the otic diseases or conditions provided herein) and may alleviate symptoms of the disease by reducing edema and/or lymphatic dysfunction.

Other compounds that both reduce edema and inhibit the lymphatic dysfunction (e.g., leaky junction associated with MD), can be used similarly to VEGF inhibitors. Steroids are not typically used as part of the treatment in the absence of a VEGF inhibitor (and sometimes are not used as part of a treatment in the absence of a VEGF inhibitor), nor are the VEGF inhibitors typically used in the same dosage as used in the treatment of cancer. The VEGF inhibitor many, in some cases, be administered in combination with other therapeutic or prophylactic agents such as steroids, diuretics, vasodilators, antiinfectives and antihistamines.

An otic disease or condition can be any appropriate otic disease or condition, such as Méniére's Disease (MD), Autoimmune Inner Ear Disease (AIED), sensorineural hearing loss (SNHL) (e.g., sudden sensorineural hearing loss (SSNHL), sensorineural hearing loss associated with diabetes, noise-induced hearing loss (NIHL), age-related hearing loss, tinnitus, or a combination thereof. In some embodiments, the otic disease or condition is not Méniére's Disease.

VEGF Inhibitors

Any appropriate VEGF inhibitor can be used in the compositions and methods provided herein. A number of therapeutic agents that inhibit VEGF are available. These include proteins such as antibodies (e.g., humanized antibodies), antibody fragments, nucleic acids such as siRNA, miRNA, triple forming compounds, DNA and mRNA and molecules that encode these inhibitors, as well as low molecular weight compounds (e.g., synthetic compounds). These typically act by binding to the VEGF receptor (e.g., competitively with a VEGF or with ATP, noncompetitively with a VEGF or with ATP, uncompetitively with a VEGF or with ATP, or allosterically), inhibiting expression of VEGF or VEGFR, or by binding to a VEGF (e.g., a decoy receptor). In some cases, a VEGF inhibitor can be an antibody or an antigen-binding fragment thereof, a decoy receptor, DNA and mRNA that encode such an antibody, antigen-binding fragment thereof, or decoy receptor, a VEGFR kinase inhibitor, an allosteric modulator of a VEGFR, or a combination thereof. In some cases, a VEGF inhibitor can be an antibody or an antigen-binding fragment thereof. For example, in some embodiments, a VEGF inhibitor can be alacizumab, bevacizumab (AVASTIN®), icrucumab (IMC-18F1), ramucirumab (LY3009806, IMC-1121B, CYRAMZA®), or ranibizumab (LUCENTIS®). In some embodiments, a VEGF inhibitor can be a decoy receptor (e.g., aflibercept). In some embodiments, a VEGF inhibitor can be a VEGFR kinase inhibitor, such as altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, or vandetanib. Other examples of VEGF inhibitors may be known in the art. In some embodiments, a VEGFR inhibitor can be an allosteric modulator of a VEGFR (e.g., cyclotraxin B).

These compounds generally have at least one of two principle mechanisms of action in treating or alleviating the symptoms of otic (e.g., inner ear) diseases or conditions: (i) inhibiting angiogenesis (e.g., vascular remodeling) and/or (ii) restoring normal vascular permeability (e.g., decreasing fluid flow through leaky junctions and cell walls and/or increasing vascular integrity), thereby reducing edema or swelling and/or restoring the ionic composition of the endolymph and perilymph.

The VEGF family includes multiple family members that are involved in the normal development of the vasculature and lymphatics. The human VEGF family includes five related proteins: VEGFA, VEGFB, VEGFC, VEGFD, and PIGF (placental growth factor). These can be secreted and form homodimers, and can interact with the VEGFR family of receptor tyrosine kinases including VEGFR1 (VEGF receptor 1), VEGFR2 (VEGF receptor 2), and VEGFR3 (VEGF receptor 3). VEGFA and VEGFB can bind to VEGFR1, VEGFA can bind to VEGFR2, and VEGFC and VEGFD can bind to both VEGFR2 and VEGFR3. PIGF primarily interacts with VEGFR1. The VEGFRs are found on a wide variety of cell types. VEGFR1, also called Flt-1 (fins-like tyrosine kinase 1), is found, for example, on vascular endothelial cells, hematopoietic stem cells, monocytes, and macrophages. VEGFR2, also called KDR (kinase insert domain) or Flk-1 (fetal liver kinase 1), is expressed on vascular and lymphatic endothelial cells; VEGFR3 (also called Flt-4) is typically found expressed on lymphatic endothelial cells. Upon ligand binding, VEGFRs transduce intracellular signals through a variety of mediators. In the case of VEGFR2, these can include phosphotidylinositol-3 kinase (PI3K)/Akt, mitogen-activated kinases (MAPKs), the nonreceptor tyrosine kinase Src, as well as PLCy (phospholipase C gamma)/PKC (protein kinase C), which promote angiogenesis, lymphangiogenesis, vascular permeability, and vascular homeostasis.

VEGF can exert an effect, for example, through the production of vasodilatory mediators. VEGF signaling through VEGFR can increase nitric oxide (NO) production. Upon VEGF binding, VEGFR2 can undergo autophosphorylation, and through PI3K/Akt signaling, can increase intracellular calcium. Acutely, this can activate calmodulin, which can bind to and can activate eNOS (endothelial NO synthase). Downstream signaling from PI3K/Akt can lead to direct phosphorylation of eNOS as well, which can provide a more sustained, calcium-independent stimulus to increase eNOS activity. VEGF signaling can also increase eNOS mRNA and protein levels, enhancing long-term eNOS expression. A resultant increase in NO production can promote vascular permeability and endothelial cell survival; NO can also diffuse to adjacent vascular smooth muscle cells and mediate endothelium-dependent vasodilation. In addition to NO, VEGF signaling can promote production of the vasodilatory prostanoid prostacyclin (PGI2) through activation of phospholipase A2 via PLCy/PKC.

T-cells can release various pro-angiogenic paracrine factors (including angiogenin, vascular endothelial growth factor (VEGF), fibroblast growth factor (FGF), and transforming growth factor-β (TGF-β). These can stimulate endothelial cell proliferation, migration and invasion resulting in new vascular structures sprouting from nearby blood vessels. Cell adhesion molecules, such as integrins, are important to the attachment and migration of endothelial cells to the extracellular matrix.

Angiogenesis is regulated, for example, by the activity of endogenous stimulators and inhibitors. Endogenous inhibitors of angiogenesis are involved in the day-to-day process of regulating blood vessel formation. Endogenous inhibitors are often derived from the extracellular matrix or basement membrane proteins and function by interfering with endothelial cell formation and migration, endothelial tube morphogenesis, and down-regulation of genes expressed in endothelial cells.

The vascular endothelial growth factor (VEGF) signaling pathway has been linked to a number of diseases and disorders, including cancer, rheumatoid arthritis, and age-related macular degeneration. In general, activation of the VEGF signaling pathway typically results in angiogenesis of a tissue. The VEGF signaling pathway transmits signal through its constituent three receptors: VEGFR1 (also called Flt-1; an exemplary human VEGFR1 sequence is shown in SEQ ID NO: 1), VEGFR2 (also called KDR or Flk-1; an exemplary human VEGFR2 sequence is shown in SEQ ID NO:2), and VEGFR3 (also called Flt4; an exemplary human VEGFR3 sequence is shown in SEQ ID NO:3), and its five growth factors: VEGF-A (an exemplary human VEGF-A sequence is shown in SEQ ID NO:4), VEGF-B (an exemplary human VEGF-B sequence is shown in SEQ ID NO: 5), VEGF-C (an exemplary human VEGF-C sequence is shown in SEQ ID NO: 6), VEGF-D (an exemplary human VEGF-D sequence is shown in SEQ ID NO: 7), and PlGF (placental growth factor; an exemplary human PlGF sequence is shown in SEQ ID NO: 8). The VEGFs have different affinities for the various VEGFRs; see, e.g., Shibuya, Masabumi. “VEGF-VEGFR signals in health and disease.” Biomolecules & Therapeutics 22.1 (2014): 1, doi: 10.4062/biomolther.2013.113). Both the VEGFs and the VEGFRs have variant isoforms and/or may be processed into a mature form as compared to the sequences shown herein. VEGF-A, in particular, has several isoforms in humans. The VEGFRs can typically be spliced as soluble or membrane-bound forms. In some accounts, the VEGFRs are grouped with the platelet-derived growth factor receptors (PDGFRs) as a superfamily of tyrosine kinase receptors.

human VEGFRI sequence from Uniparc ID UPI000013DCDD,
SEQ ID NO: 1
MVSYWDTGVL LCALLSCLLL TGSSSGSKLK DPELSLKGTQ HIMQAGQTLH
LQCRGEAAHK WSLPEMVSKE SERLSITKSA CGRNGKQFCS TLTLNTAQAN
HTGFYSCKYL AVPTSKKKET ESAIYIFISD TGRPFVEMYS EIPEIIHMTE
GRELVIPCRV TSPNITVTLK KFPLDTLIPD GKRIIWDSRK GFIISNATYK
EIGLLTCEAT VNGHLYKTNY LTHRQTNTII DVQISTPRPV KLLRGHTLVL
NCTATTPLNT RVQMTWSYPD EKNKRASVRR RIDQSNSHAN IFYSVLTIDK
MQNKDKGLYT CRVRSGPSFK SVNTSVHIYD KAFITVKHRK QQVLETVAGK
RSYRLSMKVK AFPSPEVVWL KDGLPATEKS ARYLTRGYSL IIKDVTEEDA
GNYTILLSIK QSNVFKNLTA TLIVNVKPQI YEKAVSSFPD PALYPLGSRQ
ILTCTAYGIP QPTIKWFWHP CNHNHSEARC DFCSNNEESF ILDADSNMGN
RIESITQRMA IIEGKNKMAS TLVVADSRIS GIYICIASNK VGTVGRNISF
YITDVPNGFH VNLEKMPTEG EDLKLSCTVN KFLYRDVTWI LLRTVNNRTM
HYSISKQKMA ITKEHSITLN LTIMNVSLQD SGTYACRARN VYTGEEILQK
KEITIRDQEA PYLLRNLSDH TVAISSSTTL DCHANGVPEP QITWFKNNHK
IQQEPGIILG PGSSTLFIER VTEEDEGVYH CKATNQKGSV ESSAYLTVQG
TSDKSNLELI TLTCTCVAAT LFWLLLTLFI RKMKRSSSEI KTDYLSIIMD
PDEVPLDEQC ERLPYDASKW EFARERLKLG KSLGRGAFGK VVQASAFGIK
KSPTCRTVAV KMLKEGATAS EYKALMTELK ILTHIGHHLN VVNLLGACTK
QGGPLMVIVE YCKYGNLSNY LKSKRDLFFL NKDAALHMEP KKEKMEPGLE
QGKKPRLDSV TSSESFASSG FQEDKSLSDV EEEEDSDGFY KEPITMEDLI
SYSFQVARGM EFLSSRKCIH RDLAARNILL SENNWKICD FGLARDIYKN
PDYVRKGDTR LPLKWMAPES IFDKIYSTKS DVWSYGVLLW EIFSLGGSPY
PGVQMDEDFC SRLREGMRMR APEYSTPEIY QIMLDCWHRD PKERPRFAEL
VEKLGDLLQA NVQQDGKDYI PINAILTGNS GFTYSTPAFS EDFFKESISA
PKFNSGSSDD VRYVNAFKFM SLERIKTFEE LLPNATSMFD DYQGDSSTLL
ASPMLKRFTW TDSKPKASLK IDLRVTSKSK ESGLSDVSRP SFCHSSCGHV
SEGKRRFTYD HAELERKIAC CSPPPDYNSV VLYSTPPI
human VEGFR2 sequence from Uniparc entry UPI000003AE04,
SEQ ID NO: 2
MQSKVLLAVA LWLCVETRAA SVGLPSVSLD LPRLSIQKDI LTIKANTTLQ
ITCRGQRDLD WLWPNNQSGS EQRVEVTECS DGLFCKTLTI PKVIGNDTGA
YKCFYRETDL ASVIYVYVQD YRSPFIASVS DQHGVVYITE NKNKTVVIPC
LGSISNLNVS LCARYPEKRF VPDGNRISWD SKKGFTIPSY MISYAGMVFC
EAKINDESYQ SIMYIVVVVG YRIYDVVLSP SHGIELSVGE KLVLNCTART
ELNVGIDFNW EYPSSKHQHK KLVNRDLKTQ SGSEMKKFLS TLTIDGVTRS
DQGLYTCAAS SGLMTKKNST FVRVHEKPFV AFGSGMESLV EATVGERVRI
PAKYLGYPPP EIKWYKNGIP LESNHTIKAG HVLTIMEVSE RDTGNYTVIL
TNPISKEKQS HVVSLVVYVP PQIGEKSLIS PVDSYQYGTT QTLTCTVYAI
PPPHHIHWYW QLEEECANEP SQAVSVTNPY PCEEWRSVED FQGGNKIEVN
KNQFALIEGK NKTVSTLVIQ AANVSALYKC EAVNKVGRGE RVISFHVTRG
PEITLQPDMQ PTEQESVSLW CTADRSTFEN LTWYKLGPQP LPIHVGELPT
PVCKNLDTLW KLNATMFSNS TNDILIMELK NASLQDQGDY VCLAQDRKTK
KRHCVVRQLT VLERVAPTIT GNLENQTTSI GESIEVSCTA SGNPPPQIMW
FKDNETLVED SGIVLKDGNR NLTIRRVRKE DEGLYTCQAC SVLGCAKVEA
FFIIEGAQEK TNLEIIILVG TAVIAMFFWL LLVIILRTVK RANGGELKTG
YLSIVMDPDE LPLDEHCERL PYDASKWEFP RDRLKLGKPL GRGAFGQVIE
ADAFGIDKTA TCRTVAVKML KEGATHSEHR ALMSELKILI HIGHHLNVVN
LLGACTKPGG PLMVIVEFCK FGNLSTYLRS KRNEFVPYKT KGARFRQGKD
YVGAIPVDLK RRLDSITSSQ SSASSGFVEE KSLSDVEEEE APEDLYKDFL
TLEHLICYSF QVAKGMEFLA SRKCIHRDLA ARNILLSEKN VVKICDFGLA
RDIYKDPDYV RKGDARLPLK WMAPETIFDR VYTIQSDVWS FGVLLWEIFS
LGASPYPGVK IDEEFCRRLK EGTRMRAPDY TTPEMYQTML DCWHGEPSQR
PTFSELVEHL GNLLQANAQQ DGKDYIVLPI SETLSMEEDS GLSLPTSPVS
CMEEEEVCDP KFHYDNTAGI SQYLQNSKRK SRPVSVKTFE DIPLEEPEVK
VIPDDNQTDS GMVLASEELK TLEDRTKLSP SFGGMVPSKS RESVASEGSN
QTSGYQSGYH SDDTDTTVYS SEEAELLKLI EIGVQTGSTA QILQPDSGTT
LSSPPV
human VEGFR3 sequence from Uniparc entry UPI00001488E7,
SEQ ID NO: 3
MQRGAALCLR LWLCLGLLDG LVSGYSMTPP TLNITEESHV IDTGDSLSIS
CRGQHPLEWA WPGAQEAPAT GDKDSEDTGV VRDCEGTDAR PYCKVLLLHE
VHANDTGSYV CYYKYIKARI EGTTAASSYV FVRDFEQPFI NKPDTLLVNR
KDAMWVPCLV SIPGLNVTLR SQSSVLWPDG QEVVWDDRRG MLVSTPLLHD
ALYLQCETTW GDQDFLSNPF LVHITGNELY DIQLLPRKSL ELLVGEKLVL
NCTVWAEFNS GVTFDWDYPG KQAERGKWVP ERRSQQTHTE LSSILTIHNV
SQHDLGSYVC KANNGIQRFR ESTEVIVHEN PFISVEWLKG PILEATAGDE
LVKLPVKLAA YPPPEFQWYK DGKALSGRHS PHALVLKEVT EASTGTYTLA
LWNSAAGLRR NISLELVVNV PPQIHEKEAS SPSIYSRHSR QALTCTAYGV
PLPLSIQWHW RPWTPCKMFA QRSLRRRQQQ DLMPQCRDWR AVTTQDAVNP
IESLDTWTEF VEGKNKTVSK LVIQNANVSA MYKCVVSNKV GQDERLIYFY
VTTIPDGFTI ESKPSEELLE GQPVLLSCQA DSYKYEHLRW YRLNLSTLHD
AHGNPLLLDC KNVHLFATPL AASLEEVAPG ARHATLSLSI PRVAPEHEGH
YVCEVQDRRS HDKHCHKKYL SVQALEAPRL TQNLTDLLVN VSDSLEMQCL
VAGAHAPSIV WYKDERLLEE KSGVDLADSN QKLSIQRVRE EDAGRYLCSV
CNAKGCVNSS ASVAVEGSED KGSMEIVILV GTGVIAVFFW VLLLLIFCNM
RRPAHADIKT GYLSIIMDPG EVPLEEQCEY LSYDASQWEF PRERLHLGRV
LGYGAFGKVV EASAFGIHKG SSCDTVAVKM LKEGATASEH RALMSELKIL
IHIGNHLNVV NLLGACTKPQ GPLMVIVEFC KYGNLSNFLR AKRDAFSPCA
EKSPEQRGRF RAMVELARLD RRRPGSSDRV LFARFSKTEG GARRASPDQE
AEDLWLSPLT MEDLVCYSFQ VARGMEFLAS RKCIHRDLAA RNILLSESDV
VKICDFGLAR DIYKDPDYVR KGSARLPLKW MAPESIFDKV YTTQSDVWSF
GVLLWEIFSL GASPYPGVQI NEEFCQRLRD GTRMRAPELA TPAIRRIMLN
CWSGDPKARP AFSELVEILG DLLQGRGLQE EEEVCMAPRS SQSSEEGSFS
QVSTMALHIA QADAEDSPPS LQRHSLAARY YNWVSFPGCL ARGAETRGSS
RMKTFEEFPM TPTTYKGSVD NQTDSGMVLA SEEFEQIESR HRQESGFSCK
GPGQNVAVTR AHPDSQGRRR RPERGARGGQ VFYNSEYGEL SEPSEEDHCS
PSARVTFFTD NSY
human VEGF-A sequence from Uniparc entry UPI0000030866,
SEQ ID NO: 4
MNFLLSWVHW SLALLLYLHH AKWSQAAPMA EGGGQNHHEV VKFMDVYQRS
YCHPIETLVD IFQEYPDEIE YIFKPSCVPL MRCGGCCNDE GLECVPTEES
NITMQIMRIK PHQGQHIGEM SFLQHNKCEC RPKKDRARQE KKSVRGKGKG
QKRKRKKSRY KSWSVYVGAR CCLMPWSLPG PHPCGPCSER RKHLFVQDPQ
TCKCSCKNTD SRCKARQLEL NERTCRCDKP RR
human VEGF-B sequence from Uniparc entry UPI0000001047,
SEQ ID NO: 5
MSPLLRRLLL AALLQLAPAQ APVSQPDAPG HQRKVVSWID VYTRATCQPR
EVVVPLTVEL MGTVAKQLVP SCVTVQRCGG CCPDDGLECV PTGQHQVRMQ
ILMIRYPSSQ LGEMSLEEHS QCECRPKKKD SAVKPDRAAT PHHRPQPRSV
PGWDSAPGAP SPADITHPTP APGPSAHAAP STTSALTPGP AAAAADAAAS
SVAKGGA
human VEGF-C sequence from Uniparc entry UPI0000001C2A,
SEQ ID NO: 6
MHLLGFFSVA CSLLAAALLP GPREAPAAAA AFESGLDLSD AEPDAGEATA
YASKDLEEQL RSVSSVDELM TVLYPEYWKM YKCQLRKGGW QHNREQANLN
SRTEETIKFA AAHYNTEILK SIDNEWRKTQ CMPREVCIDV GKEFGVATNT
FFKPPCVSVY RCGGCCNSEG LQCMNTSTSY LSKTLFEITV PLSQGPKPVT
ISFANHTSCR CMSKLDVYRQ VHSIIRRSLP ATLPQCQAAN KTCPTNYMWN
NHICRCLAQE DFMFSSDAGD DSTDGFHDIC GPNKELDEET CQCVCRAGLR
PASCGPHKEL DRNSCQCVCK NKLFPSQCGA NREFDENTCQ CVCKRTCPRN
QPLNPGKCAC ECTESPQKCL LKGKKFHHQT CSCYRRPCTN RQKACEPGFS
YSEEVCRCVP SYWKRPQMS
human VEGF-D sequence from Uniparc entry UPI00000012B2,
SEQ ID NO: 7
MYREWVVVNV FMMLYVQLVQ GSSNEHGPVK RSSQSTLERS EQQIRAASSL
EELLRITHSE DWKLWRCRLR LKSFTSMDSR SASHRSTRFA ATFYDIETLK
VIDEEWQRTQ CSPRETCVEV ASELGKSTNT FFKPPCVNVF RCGGCCNEES
LICMNTSTSY ISKQLFEISV PLTSVPELVP VKVANHTGCK CLPTAPRHPY
SIIRRSIQIP EEDRCSHSKK LCPIDMLWDS NKCKCVLQEE NPLAGTEDHS
HLQEPALCGP HMMFDEDRCE CVCKTPCPKD LIQHPKNCSC FECKESLETC
CQKHKLFHPD TCSCEDRCPF HTRPCASGKT ACAKHCRFPK EKRAAQGPHS
RKNP
human P1GF precursor sequence from Uniparc entry UPI0000131BEF,
SEQ ID NO: 8
MPVMRLFPCF LQLLAGLALP AVPPQQWALS AGNGSSEVEV VPFQEVWGRS
YCRALERLVD VVSEYPSEVE HMFSPSCVSL LRCTGCCGDE NLHCVPVETA
NVTMQLLKIR SGDRPSYVEL TFSQHVRCEC RHSPGRQSPD MPGDFRADAP
SFLPPRRSLP MLFRMEWGCA LTGSQSAVWP SSPVPEEIPR MHPGRNGKKQ
QRKPLREKMK PERCGDAVPR R

In some embodiments, a VEGF inhibitor can be selective for VEGFR2 over other VEGFRs. In some embodiments, VEGF inhibitor can exhibit at least a 10-fold selectivity for VEGFR2 over another VEGFR. For example, VEGF inhibitor can exhibit at least a 20-fold selectivity, at least a 30-fold selectivity, at least a 40-fold selectivity; at least a 50-fold selectivity; at least a 60-fold selectivity; at least a 70-fold selectivity; at least a 80-fold selectivity; at least a 90-fold selectivity; at least 100-fold selectivity; at least 200-fold selectivity; at least 300-fold selectivity; at least 400-fold selectivity; at least 500-fold selectivity; at least 600-fold selectivity; at least 700-fold selectivity; at least 800-fold selectivity; at least 900-fold selectivity; or at least 1000-fold selectivity for VEGFR2 over another VEGFR. In some embodiments, selectivity for VEGFR2 over another VEGFR is measured in an enzyme assay. In some embodiments, another VEGFR can be selected from the group consisting of VEGFR1, VEGFR3, and both VEGFR1 and VEGFR3.

Additional exemplary VEGF inhibitors are described at www.drugs.com/drug--class/vegf-vegfr-inhibitors.html, which is incorporated by reference in its entirety.

Angiogenic inhibitors generally reduce the production of pro-angiogenic factors, prevent them binding to their receptors, or block their actions. Compounds that inhibit the VEGF pathway include, for example, antibodies directed against VEGF or VEGFR, soluble VEGFR/VEGFR hybrids, and tyrosine kinase inhibitors that bind to one or more VEGFRs.

A widely used VEGF pathway inhibitor is bevacizumab. Bevacizumab binds to VEGFA and inhibits it from binding to VEGF receptors. Another antibody inhibitor of

VEGFA is ranibizumab. Other biologic drugs which bind to and inhibit VEGF include, for example, the VEGF decoy receptors aflibercept and conbercept.

Tyrosine Kinase Inhibitors

A number of small molecule agents, particularly tyrosine kinase inhibitors (TKIs), have been found to effectively inhibit VEGF signaling by inhibiting VEGFR. Non-limiting examples of tyrosine kinase inhibitors with known VEGFR activity include pazopanib, sunitinib, sorafenib, axitinib, regorafenib, cabozantinib, lenvatinib, nintedanib, vandetanib and apatinib. Tyrosine kinase inhibitors typically target multiple receptors. In some embodiments, an active agent as described herein is a TM with high affinity and/or specificity for VEGFR2. Other tyrosine kinase inhibitors may also be useful. Non-limiting examples of TKIs include those found at www.selleckchem.com/pharmacological receptor-tyrosine-kinase.html and www.abcam.com/products?keywords=VEGFR%20inhibitor&gclid=Cj0KCQjwivbsBRDsARIsADyISJ-Z2XBtzsvtazwh4ImDlt2YOwLuE513f2T-bWYJn-vIL_xrQXEzIF8aApHKEALw_wcB, both of which are incorporated by reference in their entireties. These VEGF inhibitors have been used, in some cases, to effectively treat cancer, macular degeneration in the eye, and other diseases that involve a proliferation of blood vessels and/or disruption of vascular wall integrity. See, e.g., “Mechanisms of VEGF (Vascular Endothelial Growth Factor) Inhibitor—Associated Hypertension and Vascular Disease” Pandev, et al. Hypertension. 2018;71:e1-e8. However, the dosages and routes of administration may be different for treatment of otic diseases or disorders (e.g., MD, AIED, SNHL (e.g., SSNHL or sensorineural hearing loss associated with diabetes), NIHL, age-related hearing loss, tinnitus, or a combination thereof), where an active agent is used to reduce edema and/or vascular dysfunction affecting the BLB.

For some otic diseases or conditions (e.g., MD), steroids, diuretics, and vasodilators are not equivalent to, nor useful in place of, VEGF inhibitors.

Additional Therapeutic Agents

In some embodiments, a VEGF inhibitor may be administered in combination with one or more other therapeutic or prophylactic agents such as steroids, diuretics, vasodilators, aminoglycosides, antiinfectives, antihistamines, or a combination thereof.

In some embodiments, a VEGF inhibitor may be administered with an inhibitor of angiopoietin-2 (and/or its receptor Tie-2), angiopoietin-1, or a combination thereof.

B. Hydrogels

In some embodiments, formulations provided herein are provided in the form of hydrogels including an active agent (e.g., a VEGF inhibitor). In some embodiments, formulations provided herein are provided in the form of a solution or suspension that forms a hydrogel in situ in a subject at a site of action.

Hydrogels are formed of networks of physically or chemically crosslinked polymers imbibed with aqueous media such as water or biological fluids. Chemical crosslinks (e.g., covalent bonds) or physical junctions (e.g., hydrophobic associations, crystallite formation, chain entanglements) generally provide the hydrogels' three-dimensional structure. As used herein, a polymer that forms a hydrogel can generally be called a “hydrogel forming agent”. In situ forming hydrogels typically occur when a polymer (e.g., hydrogel forming agent) solution is prepared and allowed to gel in situ, after photopolymerization, chemical crosslinking, ionic crosslinking or in response to an environmental stimulus such as temperature, pH or ionic strength of the surrounding medium. In some embodiments, a hydrogel forming agent can be a hydrogel forming agent that undergoes chemical crosslinking to form a hydrogel.

“Synthetic polymers” as used herein refers to polymers that are auris-acceptable. In some embodiments, a synthetic polymer can be used in a formulation provided herein, for example, as a hydrogel forming agent.

The phenomenon of transition from a solution to a gel is commonly referred to as a sol-gel transition.

A formulation may be in the form of a solution or suspension that effects a transition from a liquid state at room temperature to a hydrogel at body temperature, for example, such that the hydrogel provides sustained release of an active agent for a period of between at least three to fifteen days in the ear. In some embodiments, it is important that the formulation can be injected into the inner ear, preferably using a high-gauge needle, where it then solidifies, typically through a sol-gel transition effected by the increased temperature of the body relative to the temperature at which the formulation was prepared and/or stored.

In some instances, intra-tympanic injection of a cold composition (e.g., a composition with a temperature of <20° C.) can cause a density gradient in the inner ear fluids that induces vertigo, in individuals undergoing prevention for inner ear disorders. In some embodiments, the formulations provided herein are designed to be liquids that are administered at or near room temperature and do not cause vertigo or other discomfort when administered to an individual or patient.

Temperature Mediated Phase Transition Gels

In some embodiments, a hydrogel can be a temperature mediated phase transition gel. Hydrogels that are sensitive to thermal stimuli can be useful, as temperature is the sole stimulus for their gelation with no other requirement for chemical or environmental treatment and can be thus produced e.g., upon injection to the body, when temperature is increased from ambient to physiological. Non-limiting examples of hydrogels that are sensitive to thermal stimuli are described in U.S. Pat. No. 10,561,736 and U.S. Patent Application Publication No. 2020/0214976.

Some hydrogels exhibit a phase transition from a liquid solution to a solid hydrogel above a certain temperature. This threshold is typically defined as the lower critical solution temperature (LCST). Below the LCST, the polymers exist as single chains or are associated in unpacked micelles. Above the LCST, they become increasingly hydrophobic and insoluble, leading to gel formation. Hydrogels that are formed upon cooling of a polymer solution have an upper critical solution temperature (UCST). The sol-gel transition of thermosensitive hydrogels can be experimentally verified by a number of techniques such as the vial inversion method, spectroscopy, differential scanning calorimetry (DSC), and rheology.

Some natural polymers can transition from a liquid to a solid state based on temperature, such as some of the modified cyclodextrins. In some embodiments, a natural polymer that can transition from a liquid state to a solid state based on temperature is used in a formulation provided herein. In some embodiments, a natural polymer that can transition from a liquid state to a solid state based on temperature is not used in a formulation provided herein.

Non-limiting examples of synthetic polymers include copolymers of ethylene oxide and propylene oxide, (e.g., poloxamers (PLURONICS® (BASF)) such as POLOXAMER® 407 and POLOXAMER® 188), and that demonstrate a sol-gel transition. In some embodiments, a synthetic polymer can be a N-isopropylacrylamide (NiPAAM) polymer, a poly(ethylene oxide)-b-poly(propylene oxide)-b-poly(ethylene oxide) (PEO-PPO-PEO) polymer, a poly(ethylene glycol) (PEG)-biodegradable polyester copolymer, or a combination thereof. POLOXAMERS® include, for example, PLURONICS® F68, F88, F108, and F127 which are block copolymers of ethylene oxide and propylene oxide); and POLOXAMINES® (e.g., TETRONIC® 908, also known as POLOXAMINE® 908, which is a tetrafunctional block copolymer derived from sequential addition of propylene oxide and ethylene oxide to ethylenediamine (BASF Corporation, Parsippany, N.J.)). In some embodiments, a solution or a suspension of a synthetic polymer can transition from a liquid to a solid based on temperature.

In some embodiments, formulations provided herein contain a POLOXAMER®, which are triblock copolymers of poly(ethylene oxide) (PEO) and poly(propylene oxide) (PPO) and are available in different molecular weights and PPO/PEO ratios.

In some embodiments, a hydrogel forming agent is POLOXAMER® 407. POLOXAMER® 407 (F-127) is a nonionic polymer composed of polyoxyethylene-polyoxypropylene copolymers. Other commonly used poloxamers include 188 (e.g., F-68 grade), 237 (e.g., F-87 grade), 338 (e.g., F-108 grade). Aqueous solutions of poloxamers are stable in the presence of acids, alkalis, and metal ions. PF-127 is a commercially available poly(oxyethylene)-poly(oxypropylene) triblock copolymer of general formula E106 P70

E106, with an average molar mass of 13,000 Da. PF-127 contains approximately 70% ethylene oxide, which provides for its hydrophilicity.

Additional non-limiting suitable hydrogel-forming agents include PLURONICs (polyethylene oxide block copolymers). In some embodiments, the hydrogel forming agent is POLOXAMER® 407.

Shear Thinning Gels

In some embodiments, a hydrogel can be a shear thinning gel.

Moldable hydrogels that can be formed and processed prior to use and subsequently applied in a conformal manner can be suitable alternatives to covalent hydrogels for some applications, including, for example, local drug delivery in the body, cell carriers for tissue engineering, bone fillers or hydraulic fracturing fluids. To serve these functions, mouldable hydrogels typically exhibit viscous flow under shear stress (shear-thinning) and rapid recovery when the applied stress is relaxed (self-healing). In addition, it is extremely beneficial if the high shear viscosity is low (e.g., η≤1 Pa·s @\dot{γ}˜100 s⁻¹) for facile application through high gauge needles. These properties typically enable minitnally invasive implantation in visy) though direct injection or catheter-based delivery.

See, for example “Self-assenibled hydrogels utilizing polymer-nanoparticle interactions” by Appel, et al. Nature Communications 6, 6295 (2015), describing the preparation and application of hydrogels driven by noncovalent interactions between hydroxypropylinethylcellulose derivatives (HPMC-x) and core-shell nanoparticles (NPs). Transient and reversible interactions between the NPs and I-IPMC chains impact polymer-NP (PNP) hydrogel self-assembly, allowing for flow under applied stress and complete recovery of their structural properties when the stress is relaxed. PNP hydrogels are formulated with poly(ethylene glycol)-block-poly(lactic acid) (PEG-b-PLA) NPs to enable dual loading of a hydrophobic molecule into the PEG-b-PLA NPs and a second, hydrophilic molecule into the aqueous bulk of the gel.

See also “Smart Shear-Thinning Hydrogels as Injectable Drug Delivery Systems” Gharaie, et al. Polymers (Basel). 2018 December; 10(12): 1317. Published online 2018 Nov 28. doi: 10.3390/polym10121317

Self-Assembling Gels

In some embodiments, a hydrogel can be self-assembling gel.

Self-assembly via non-covalent crosslinking can provide a route to fabricate moldable and/or injectable hydrogels with shear-thinning and/or self-healing properties arising from strong, yet transient and reversible crosslinks. Several exemplary systems have been reported utilizing natural host—guest or receptor—ligand pairs, such as (strept)avidin with biotin, leucine zipper and ‘dock-and-lock’ protein structures prepared with genetic engineering techniques, or with synthetic macrocyclic host molecules, such as cyclodextrins or cucurbit[n]urils. See for example, Park, K. M. et al. In situ supramolecular assembly and modular modification of hyaluronic acid hydrogels for 3D cellular engineering. ACS Nano 6, 2960-2968 (2012). Appel, E. A. et al. Supramolecular cross-linked networks via host-guest complexation with cucurbit[8]uril. J. Am. Chem. Soc. 132, 14251-14260 (2010); Appel, et al

Sustained release of proteins from high water content supramolecular hydrogels. Biomaterials 33, 4646-4652 (2012).

Ionically Crosslinked Alginate Gels

In some embodiments, a hydrogel can be an ionically crosslinked gel.

In some cases, a hydrogel that is injected as a liquid and that gels when in contact with ions (e.g., calcium) can be used for delivery of an active agent. The precursors of the hydrogel, such as alginate, can be mixed with calcium ions prior to injection into the body and gel following injection, or can gel upon contact with Ca²⁺ in the body. In some cases, an ionically-crosslinked hydrogel can also be thermoresponsive. See, for example, Cui, H.; Messersmith, P. B. Thermally triggered gelation of alginate for controlled release, in tailored polymeric materials for controlled delivery systems. Amer. Chem. Soc. 1998, 203-211; Westhaus, E.; Messersmith, P. B. Triggered release of calcium from lipid vesicles: A bioinspired strategy for rapid gelation of polysaccharide and protein hydrogels. Biomaterials 2001, 22, 453-462; Cui, et al.; Thermally triggered gelation of alginate for controlled release. In Tailored Polymeric Materials For Controlled Delivery Systems; American Chemical Society: Washington, D.C., USA, 1998.

Dendrimers

In some embodiments, a hydrogel can be a dendrimer gel.

Dendrimers are generally discrete nanostructures/nanoparticles with onion skin-like branched layers. Beginning with a core, these nanostructures can grow in concentric layers to produce stepwise increases in size that are similar to the dimensions of many in vivo globular proteins. These branched tree-like concentric layers are sometimes referred to as ‘generations’. The outer generation of each dendrimer presents a precise number of functional groups that may act as a monodispersed platform for engineering favorable nanoparticle-drug and nanoparticle-tissue interactions. See “Emerging concepts in dendrimer-based nanomedicine: from design principles to clinical applications” Kannan, et al. J Intern Med. 2014 December; 276(6):579-617. doi: 10.1111/joim.12280. Epub 2014 Jul 31. It is believed that the application of dendrimers as a drug delivery system started in late 1990s. Dendrimers for drug delivery can be employed using a formulation and/or nanoconstruct approach. In the formulation approach, an active agent (e.g., one or more drugs) are physically entrapped in a dendrimer using non-covalent interactions, whereas an active agent (e.g., one or more drugs) are covalently coupled on dendrimers in the nanoconstruct approach. PAMAM dendrimers have been demonstrated to enhance solubility, stability and oral bioavailability of various active agents (e.g., drugs). Active agent (e.g., drug) entrapment and active agent (e.g., drug) release from dendrimers can be controlled by modifying dendrimer surfaces and generations. PAMAM dendrimers are also shown to increase transdermal permeation and specific active agent (e.g., drug) targeting. Dendrimer platforms can be engineered to attach targeting ligands and imaging molecules to create a nanodevice. In some embodiments, dendrimeric materials include G0-G10 PAMAM dendrimers.

Exemplary reviews of dendrimers in drug delivery include Chauhan, et al. Molecules. 2018 Apr. 18; 23(4). pii: E938. doi: 10.3390/molecules23040938. The dendrimer architecture can have three main sites for active agent (e.g., drug) entrapment by using various mechanisms: (i) void spaces (e.g., by molecular entrapment); (ii) branching points (e.g., by hydrogen bonding); and/or (iii) outside surface groups (e.g., by charge—charge interactions).

C. Delivery Vehicles and Formulations

A formulation as provided herein can be any appropriate formulation. In some embodiments, a formulation can be an injectable sustained release formulation or a sustained release device (e.g., an implant). For example, a sustained release formulation or device may include bioerodible polymer implants or injectables (such as polylactide-co-glycolide (“PLGA” or polycaprolactone (PCL)), such as micro- or nanoparticles, devices coated or wholly comprised of nonbioerodible polymer implants (such as polybutylmethacrylate (PBMA), polyethylene vinyl acetate (PEVA), or silicone), or reservoir systems which provide continuous drug delivery through active pumping or passive diffusion.

In some cases, a formulation may be administered as a solution or suspension or as a controlled or sustained release formulation. In some embodiments, a formulation can be administered by injection, for example, with the size of the needle dependent on the site of administration. For example, a smaller gauge needle would be used for intraocular administration than for administration into the middle and/or inner ear.

In some cases, a formulation may be in the form of an aqueous solution or suspension, typically buffered such as with phosphate buffered saline, or in the form of a hydrogel (e.g., a hydrogel, a shear thinning gel, a phase transition gel, a self-assembling gel, and/or a suspension of particles in a gel or aqueous solution)). In some embodiments, a formulation can be in the form of a solution or suspension that forms a hydrogel in a subject. An active agent to be released may be dispersed or dissolved in the gel or solution and/or encapsulated in or bound to particles for release. In some embodiments, particles may be formed of the therapeutic agent or polymeric materials such as poly(lactide-co-glycolide), or guest-host complexes such as dendrimers or cyclodextrin complexes.

In some embodiments, a formulation as provided herein can include at least an active agent (e.g., a VEGF inhibitor) and a hydrogel forming agent. In some embodiments, active agent is present in the formulation in an amount of about 0.0031% (w/w) to about 1.5% (w/w) (e.g., about 0.0031% (w/w) to about 0.005% (w/w), about 0.0031% (w/w) to about 0.01% (w/w), about 0.0031% (w/w) to about 0.05% (w/w), about 0.0031% (w/w) to about 0.1% (w/w), about 0.0031% (w/w) to about 0.5% (w/w), about 0.0031% (w/w) to about 1.0% (w/w), about 0.005% (w/w) to about 1.5% (w/w), about 0.01% (w/w) to about 1.5% (w/w), about 0.05% (w/w) to about 1.5% (w/w), about 0.1% (w/w) to about 1.5% (w/w), about 0.5% (w/w) to about 1.5% (w/w), or about 1.0% (w/w) to about 1.5% (w/w)) of the formulation. In some embodiments, active agent is present in the formulation in an amount of about 0.003% (w/w) to about 20% (w/w) (e.g., about 0.003% (w/w) to about 0.005% (w/w), about 0.003% (w/w) to about 0.01% (w/w), about 0.003% (w/w) to about 0.05% (w/w), about 0.003% (w/w) to about 0.1% (w/w), about 0.003% (w/w) to about 0.5% (w/w), about 0.003% (w/w) to about 1% (w/w), about 0.003% (w/w) to about 2% (w/w), about 0.003% (w/w) to about 5% (w/w), about 0.003% (w/w) to about 10% (w/w), about 0.003% (w/w) to about 15% (w/w), about 0.005% (w/w) to about 20% (w/w), about 0.01% (w/w) to about 20% (w/w), about 0.05% (w/w) to about 20% (w/w), about 0.1% (w/w) to about 20% (w/w), about 0.5% (w/w) to about 20% (w/w), about 1% (w/w) to about 20% (w/w), about 2% (w/w) to about 20% (w/w), about 5% (w/w) to about 20% (w/w), about 10% (w/w) to about 20% (w/w) about 15% (w/w) to about 20% (w/w), about 0.01% (w/w) to about 10% (w/w), about 0.5% (w/w) to about 10% (w/w), about 1% (w/w) to about 15% (w/w), about 5% (w/w) to about 15% (w/w), about 7% (w/w) to about 12% (w/w), about 8% (w/w) to about 11% (w/w), or about 9% (w/w) to about 10% (w/w)) of the formulation. In some embodiments, the hydrogel forming agent is present in the formulation in an amount of about 5% to 30% (e.g., about 5% to about 10%, about 5% to about 15%, about 5% to about 20%, about 5% to about 25%, about 10% to about 12%, about 10% to about 15%, about 10% to about 20%, about 10% to about 25%, about 10% to about 30%, about 12% to about 30%, about 15% to about 30%, about 20% to about 30%, about 25% to about 30%, about 8% to about 12%, or about 9% to about 10%) by weight of the formulation. In some embodiments, the hydrogen forming agent is present in an amount of about 8% to about 12% by weight of the formulation. In some embodiments, the hydrogen forming agent is present in an amount of about 9% to about 10% by weight of the formulation. In some embodiments, the hydrogen forming agent is present in an amount of about 15% by weight of the formulation. In some embodiments, the hydrogel forming agent is POLOXAMER® 407.

Formulations as provided herein can include any appropriate additives or other components. In some embodiments, a formulation can further include one or more of sodium chloride, water, an antioxidant, an antimicrobial, a detergent, a solubilizing agent, a stabilizer, a crystallization inhibitor, a viscosity modifier, a chelator, or a buffer (e.g., a phosphate buffer (e.g., hydrogen phosphate di-sodium dodecahydrate and dihydrogen sodium phosphate dehydrate), a Tris buffer, or a HEPES buffer). In some forms, synthetic polymers are included in a formulation provided herein to enhance physical stability or for other purposes. Additional non-limiting examples of synthetic polymers include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40;

polysorbates such as polyethylene glycol sorbitan monostearate and polyethylene glycol sorbitan monooleate; triacetin; D-a-tocopheryl polyethylene glycol succinate (vitamin E TPGS); phospholipids; lecithins; phosphatidyl cholines (c8-c18); phosphatidylethanolamines (c8-c18); phosphatidylglycerols (c8-c18); bile salts; glyceryl monostearate; polyoxyethylene fatty acid glycerides; vegetable oils such as polyoxyethylene (60) hydrogenated castor oil; and polyoxyethylene alkylethers and alkylphenyl ethers such as octoxynol 10, octoxynol 40; or a combination thereof.

Formulations as provided herein can have any appropriate properties. In some embodiments, a formulation can have a viscosity suitable for injection through a 23-G needle, for example, for injection through the tympanic membrane into the tympanic cavity.

In some embodiments, a hydrogel formed from a formulation provided herein can provide, for example, sustained release of an active agent for a period of at least 3-15 days in the ear. In some embodiments, a hydrogel formed from such a formulation can provide, for example, sustained release of an active agent for an extended period of time (e.g., one day, 2 days, 3 days, 4 days, 5 days, 6 days, 1 week, 2 weeks, 3 weeks, 1 month, 2 months, 3 months, 4 months, 5 months, or 6 months).

D. Other Additives and Excipients

Formulations as provided herein can contain one or more additional components such as a pH buffer, a tonicity agent, a mucoadhesive agent, a stabilizing agent, a preservative, a carrier, a viscosity enhancing agent, a penetration enhancer, or a combination thereof.

In some embodiments, other materials can be incorporated into the hydrogel forming agent, or the solution or suspension of the hydrogel forming agent. Representative materials include diluents, buffers, dispersing agents or viscosity modifying agents, solubilizers, stabilizers, and osmolarity modifying agents. The pH of a formulation is, in some embodiments, between 6.8 and 7.7, for example 7.2. In some embodiments, a formulation provided herein has an osmolality of about 280 mOsmol/kg. The term “diluent” refers to agents (e.g., chemical compounds) that are used to dilute, preferably, the otic agent prior to delivery, and which are compatible, preferably, with the auris media and/or auris interna.

The terms “dispersing agents,” and/or “viscosity modulating agents” and/or “viscosity modifiers” and/or “thickening agents” refer to materials that can enhance dispersion of particulate matter in a solution and/or modify the viscosity of a solution or suspension. Examples of dispersing agents/materials include, but are not limited to, hydrophilic polymers, electrolytes, TWEEN® 60 or TWEEN® 80, PEG,polyvinylpyrrolidone (PVP; also known as povidone and commercially known as Kollidon®, and PLASDONE®) , and the carbohydrate-based dispersing agents such as, for example, modified celluloses such as hydroxypropyl celluloses (e.g., HPC, HPC-SL, and HPC-L), hydroxypropyl methylcelluloses (e.g., HPMC K100, HPMC K4M, HPMC K15M, and HPMC K100M),carboxymethylcellulose, carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), and polyethylene glycol (e.g., having a molecular weight of about 300 to about 6000, or about 3350 to about 4000, or about 7000 to about 5400). In some embodiments, the amount of thickening agent is about 1% to about 15% (e.g., about 1% to about 10%, about 1% to about 5%, about 5% to about 15%, about 10% to about 15%, 1%, 5%, about 10%, or about 15%) of the total weight of a formulation. In some instances, dispersants improve formulation stability by inhibiting drug crystallization.

In some embodiments, a formulation provided herein can have a suitable viscosity for injection through a 23-G needle or a needle of a higher gauge. At elevated temperatures (above 26° C.), the viscosity can increase (for example, due to the sol-gel transition) to above 100,000 cP. At 14.73 w/w P407, the viscosity is about 100 cP at temperatures below 20° C.

The term “solubilizer” can refer to auris-acceptable compounds such as triacetin, triethylcitrate, ethyl oleate, ethyl caprylate, sodium lauryl sulfate, sodium doccusate, vitamin

E TPGS, dimethylacetamide, N-methylpyrrolidone, N-hydroxyethylpyrrolidone, polyvinylpyrrolidone, hydroxypropylmethyl cellulose, hydroxypropyl cyclodextrins and other cyclodextrins, ethanol, n-butanol, isopropyl alcohol, cholesterol, bile salts, polyethylene glycol 200-600, glycofurol, TRANSCUTOL®, propylene glycol, and dimethyl isosorbide, ethanol, and other organic solvents. In some embodiments, a solubilizer is a propylene glycol, a polyethtylene glycol (e.g., PEG300), ethanol, or a cyclodextrin.

The term “stabilizer” can refer to compounds such as antioxidants, buffers, acids, and preservatives that are compatible, preferably, with the environment of the auris media and/or auris interna. Stabilizers include agents that improve the compatibility of excipients with a container, or a delivery system, such as a syringe or a glass bottle, improve the stability of a component of the formulation, or improve formulation overall stability.

Tonicity and pH adjusting agents may also be included in a formulation, in some cases. In general, the endolymph has a higher osmolality than the perilymph. For example, the endolymph typically has an osmolality of about 304 mOsm/kg H₂O, while the perilymph typically has an osmolality of about 294 mOsm/kg H₂O. In some forms, a formulation is formulated to provide an osmolality between about 100 mOsm/kg and about 500 mOsm/kg, between about 200 mOsm/kg and about 400 mOsm/kg, between about 240 mOsm/kg and about between 350 mOsm/kg, between about 250 mOsm/kg and about 350 mOsm/kg, between about 270 mOsm/kg and about 320 mOsm/kg, or between about 280 mOsm/kg and about 320 mOsm/kg. Osmolarity/osmolality can be adjusted, for example, by the use of appropriate salt concentrations (e.g., concentration of potassium salts) or the use of tonicity agents, which can render the compositions endolymph-compatible and/or perilymph-compatible (e.g., isotonic with the endolymph and/or perilymph). In some instances, the formulations, preferably endolymph-compatible and/or perilymph-compatible formulations, cause minimal disturbance to the environment of the inner ear and cause minimum discomfort (e.g., vertigo and/or nausea) to a subject (e.g., a mammal) upon administration.

In some cases, a formulation is isotonic with the perilymph. Isotonic formulations are typically provided by the addition of a tonicity agent. Suitable tonicity agents include, but are not limited to, a pharmaceutically acceptable sugar, salt or any combinations or mixtures thereof, such as, but not limited to dextrose, glycerin, mannitol, sorbitol, sodium chloride, and other electrolytes. Sodium chloride or other tonicity agents can be optionally used to adjust tonicity, if necessary. Representative salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. In some embodiments, a salt is sodium chloride.

Formulations provided herein can include one or more pH-adjusting agents or buffering agents. Exemplary suitable pH adjusting agents or buffers include acetate, bicarbonate, ammonium chloride, citrate, phosphate, pharmaceutically acceptable salts thereof and combinations or mixtures thereof. Non-limiting suitable water-soluble buffering agents are alkali or alkaline earth metal carbonates, phosphates, bicarbonates, citrates, borates, acetates, succinates and the like, such as sodium phosphate, citrate, borate, acetate, bicarbonate, carbonate and tromethamine (TRIS).

In some embodiments, a tbrmulation can include a rnucoadhesive. In some cases, the inucoadhesive facilitates adhesion to a portion of the ear, such as the external mucous layer of the round window membrane. Mueoadhesive agents include, but are not limited to, carbomers, such as CARBOPOL® 934P, polyvinylpyrrolidone polymer (PVP); a water-swellable, but water-insoluble, fibrous, cross-linked carboxy-functional polymer; a crosslinked poly(acric acid) (e.g. CARBOPOL® 947P); a carbomer homopolymer; carbomer copolymer; a hydrophilic polysaccharide gun); maltodextrin; a cross-linked alginate gum gel, hydroxypropyl methylcellulose, and a water-dispersible polycarboxylated vinyl polymer. Some exemplary mucoadhesive agents are described in U.S. Pat. No. 8,828,980 to Lichter, et al.

In some embodiments, a formulation can include a surfactant. Examples of surfactants include, but are not limited to, sodium lauryl sulfate, sodium decussate, TWEEN® 60 (polyethylene glycol sorbitan monostearate) or TWEEN®80 (polyethylene glycol sorbitan monooleate), triacetin, D-α-tocopheryl polyethylene glycol succinate (vitamin E TPGS), phospholipids, lecithins, phosphatidyl cholines (c8-c18), phosphatidylethanolamines (c8-c18), phosphatidylglycerols (c8-c18), sorbitan monooleate, polyoxyethylene sorbitan monooleate, polysorbates, bile salts, and glyceryl monostearate.

A formulation may include penetration enhancers that allow, for example, delivery of the agents across a barrier, such as the oval window or the round window of the ear. Typically, the penetration enhancers are auris-compatible. Exemplary penetration enhancers include sodium lauryl sulfate, sodium octyl sulfate, sodium dodecyl sulfate, ocyll-trimethyl-ammonium bromine, dodecyl-trimethyl ammonium bromide, sodium laurate, polyoxyethylene-20-cetyl ether, laureth-9, sodium dodecylsulfate, dioctyl sodium sulfosuccinate, polyoxyethylene-9-lauryl ether (PLE), TWEEN® 20, TWEEN®, 80, nonylphenoxypolyethylene (NP-POE), polysorbates, bile salts, fatty acids and derivatives, chelating agents (such as EDTA, citric acid, and salicylates, sulfoxides (such as dimethyl sulfoxide (DMSO) and decylmethyl sulfoxide), and alcohols (such as ethanol, isopropanol, glycerol, and propanediol.

In some forms, a formulation can include a preservative. Suitable preservatives include, but are not limited to, benzoic acid, boric acid, p-hydroxybenzoates, alcohols, quaternary compounds, stabilized chlorine dioxide, mercurials, such as merfen and thiomersal, or a combination thereof. Exemplary preservatives are described in U.S. Pat. No. 8,828,980 to Lichter, et al.

A formulation as provided herein can have any appropriate pH. In some embodiments, the pH of a formulation is about 6.0 to about 8.0 (e.g., about 6.0 to about 6.5, about 6.0 to about 7.0, about 6.0 to about 7.5, about 6.5 to about 8.0, about 7.0 to about 8.0, about 7.5 to about 8.0, about 6.0 to about 7.6, about 6.8 to about 7.5, about 7.0 to about 7.4, about 7,1, about 7.2, or about 7.3).

In some embodiments, a formulation can be prepared and stored in vials, syringes, capsules, ampules, or pouches prior to administration. In some embodiments, a formulation may be packaged in a single-dose that is administered intra-tympanically into the middle ear.

Formulations may be lyophilized, micronized, pelleted, or in a solution or suspension. Optionally, the components of the formulation can be provided a kit that contains instructions to formulate the composition by adding, e.g., diluent to a hydrogel-forming agent and/or active agent (e.g. VEGF inhibitor).

III. Methods of Preparing

Formulations as provided herein can be prepared by any appropriate method. Without being bound by any particular theory, it is believed that in some cases, it may be beneficial to prepare a formulation as provided herein (e.g., a hydrogel) at or near the time of administration. For example, it may be beneficial so that a formulation can be administered as a liquid, and form a hydrogel at a site of action (e.g., in an ear) of a subject.

In some embodiments, a formulation is prepared by mixing an effective amount of an active agent (e.g., a VEGF inhibitor), with one or more excipients. In some cases, the mixture can be stirred or shaken to dissolve the active agent or form a uniform suspension with the aid of suitable suspending or viscosity enhancing agents. In some embodiments, additional excipients such as buffers, salts, and preservatives can subsequently be added to the active agent. In some forms, the agent is suspended if it is insoluble in water. If needed, the pH can be modulated by the addition of appropriate buffering agents. Depending on the concentration of the active agent, it can, in some embodiments, exist as micronized particles in the composition. In some embodiments, a phosphate buffer is prepared and sterile filtered to form a solution or suspension.

In some embodiments, a method of preparing a formulation includes combining a hydrogel-forming agent, an active agent, and water. In some embodiments, prior to introducing the active agent (e.g., VEGF inhibitor), the hydrogel forming agent (e.g., POLOXAMER® 407) is formulated as a liquid product including an amount (or, e.g., concentration) of the hydrogel forming agent (e.g., POLOXAMER® 407) that at body temperature forms a hydrogel providing sustained release of the active agent. In some embodiments, the active agent(s) is then added to the liquid product. In some embodiments, this forms a homogeneous solution (e.g., without causing gelation).

IV. Methods and Routes of Administration

Active agents that bind to VEGF or a VEGF receptor (e.g., VEGF inhibitors), for example, tyrosine kinase inhibitors or compounds that reduce both edema and the lymphatic dysfunction associated with various otic diseases and conditions (e.g., MD, AIED, SNHL (e.g., SSNHL or sensorineural hearing loss associated with diabetes), NIHL, age-related hearing loss, tinnitus, or a combination thereof), (e.g. anti-VEGF antibodies or tyrosine kinase inhibitor small molecules), can be locally, regionally or systemically administered to an individual with an otic disease or condition (e.g., MD, AIED, SNHL (e.g., SSNHL or sensorineural hearing loss associated with diabetes), NIHL, age-related hearing loss, tinnitus, or a combination thereof), for example, to alleviate symptoms of the disease by reducing edema and/or lymphatic dysfunction. In some embodiments, an effective amount of these compounds is delivered by transtympanic or intracochlear administration. In some embodiments, an effective amount of these compounds to alleviate one or more symptoms of the disease or disorder is preferably delivered by intranstympanic or intracochlear administration Other methods of administration include, but are not limited to, parenteral, subcutaneous, intraperitoneal, intranasal, intravenous and oral. In some cases, a formulation may be for immediate release, sustained or controlled release. Non-limiting examples of dosage forms include solutions, suspensions, ointments, hydrogels, liposomes, controlled release particles, tablets and capsules. Delivery of an active agent (e.g., VEGF inhibitor) may also be by implantation of devices delivering the active agent. Systemic administration of VEGF inhibitors is typically not performed, since a dose required to adequately reach targets in the cochlea and vestibular system of the inner ear may be very high and may cause systemic side effects. In normal physiology, the blood-labryinth barrier (BLB) in the stria vascularis controls exchange between blood and the interstitial space in the cochlea and presents a barrier to entry of many systemically administered drugs. However, some cases, particularly conditions where vascular permeability is increased, this barrier may be compromised and permit entry of drug to a target tissue. Side effects of systemic anti-VEGF treatment are well known from oncology and can include thromboembolic events, myocardial infarction, stroke, hypertension, gastrointestinal perforations and kidney disease.

Transtympanic injection, or administration into the middle ear, can provide more direct delivery of a VEGF inhibitor than systemic administration. In this case, entry into the cochlea and vestibular system would be via diffusion through the round window membrane and/or the oval window. Recent work indicates that large molecules such as BDNF (27 kDa) may be able to distribute to the cochlea from the middle ear. Therefore, it is believed that anti-VEGF biological agents can penetrate via this route of administration. Without being bound by any particular theory, it is further believed that smaller antibody fragments such as ranibizumab (48 kDa) will likely penetrate with greater efficiency than full length antibodies such as bevacizumab (150 kDa). The greatest transport efficiency is expected with smaller molecules such as tyrosine kinase inhibitors (<1 kDa).

Direct intracochlear delivery has the advantage of bypassing membrane mediated transport. Without being bound by any particular theory, it is believed that direct intracochlear delivery would result in the lowest possible doses with greatest distribution to target tissues.

In some embodiments, a formulation (e.g., a hydrogel), device, or other delivery system releasing VEGF inhibitors can be placed in the middle ear by surgically opening the middle ear.

In some cases, treating an otic disease or condition (and/or symptoms thereof) acutely may not require sustained delivery formulations and simple injectable solutions/suspensions may be adequate. However, many otic diseases or conditions can be chronic disorder and in order to continually manage vascular integrity and prevent episodic flare-ups of the disease, more sustained delivery formulations may be desirable.

Formulations can be administered as needed based on the patient. In some cases, disease may be transient, in others, chronic. In some cases, short treatments could be administered once or twice a month, for a period of one to three months. In some cases, chronic disease could be treated once or twice a month for a period of at least one year.

A. Administration into the Ear

Formulations are typically administered to the middle and/or inner ear of a subject in need thereof. In general, methods of use involve administering to the subject (e.g., by injection, such as intratympanic injection) compositions containing an effective amount of the active agent(s).

In some cases, a formulation can also be used to treat other diseases which require a longer duration for treatment, such as through administration via a reservoir or depot.

In some embodiments, a formulation can be administered via localized administrations by intra-tympanic injection of the formulation as a solution or suspension (e.g., at room temperature or lower). Such administration routes and appropriate formulations therefor are generally known to those of skill in the art. After administration, a formulation may effect a transition from a liquid state at room temperature to a gel state at body temperature. In some embodiments, the gel state provides sustained release of the agent.

In some embodiments, a formulation may also be administered on or near the round window or the crista fenestrae cochleae through entry via a post-auricular incision and surgical manipulation into or near the round window or the crista fenestrae cochleae area.

In some embodiments administration is made using a syringe and small gauge needle, 23 G to 30 G or higher gauge, such as when a needle is inserted through the tympanic membrane. In some such embodiments, the formulation can fill the hypotympanum of the tympanic cavity, and contact the round window membrane.

In some cases, a formulation can also be administered into the tympanic cavity or applied on the tympanic membrane or onto or in the cochlea by injection, direct instillation or perfusion of the inner ear compartments, for example, via surgical procedures. In some instances, a formulation may be directly injected into the cochlea, for example, via injection through the round window membrane or a cochleostomy drilled in the bone of the cochlea.

In other embodiments, a formulation is administered via microcathethers implanted into the subject, using a drug delivery device such as a micropump, a microinjection device, or a microreservoir implanted within the inner ear.

Formulations can be administered in a single dose or in multiple doses. Certain factors may influence the dosage required to effectively treat or prevent a disorder, including, but not limited to, the severity of the disease or disorder, previous preventions, the general health and/or age of the subject, and other diseases present. It will also be appreciated that the effective dosage of the composition used for prevention may increase or decrease over the course of a particular treatment time period. Need for changes in dosage quantity or strength may result become apparent from the results of assays, for example, frequency of vertigo attacks, tinnitus, the auditory brainstem response, distortion product otoacoustic emission, word recognition scores, and/or subjective changes in balance or hearing reported by the patient.

For the calculation of the maximum recommended starting dose (MRSD), several parameters can be considered. There is currently no published reference of the total volume of perilymph volume in rats. However, the cochlear volume of perilymph in rats has been reported to be 2.63 μl (ST_{perilymph}+SV_{perilymph}=1.04+1.59=2.63 μl, ST: scala tympani; SV: scala vestibule) as described in Thorne, et al., Laryngoscop 109(10), 1661-8 (1999). The volume of perilymph in the rat semicircular canals was estimated. A cylinder shape was assumed for the semicircular canals following the method described in Buckingham and Valvassori, Ann. Otol. Rhinol. Laryngol. 110(2), 113-7 (2001) for humans and the dimensions for the semicircular canals in rat described in Cummins, J. Comp. Neurol. 38, 399-459 (1925). The total volume has been calculated as: V=π×r²×l (r: radius; l: length). The estimated volume for each canal is:

Anterior: 0.33 mm³ (π×0.125²×6.8): 0.33 μl Posterior: 0.31 mm³ (π×0.13²×6): 0.31 μl Lateral: 0.26 mm³ (π×0.125²×5.4): 0.26 μl

The total perilymph volume in rat will be then: (Cochlear_{perilymph}+Semicircular canals_{perilymph})=ST_{perilymph}+SV_{perilymph}+Semicircular canals_{perilymph}=1.04+1.59+0.9=3.53 μl

The total perilymph volume in human has been described to be 158 μl (Buckingham and Valvassori, Ann. Otol. Rhinol. Laryngol. 2001, 110(2), 113-7).

In some cases, the amount of a formulation to be injected in humans is normalized with the volumes of perilymph in rats and humans and the amount injected at the NOAEL dose as follows:

agent injected,ᵣₐₜₛ/Volume perilymphᵣₐₜₛ=agent injected,ₕᵤₘₐₙₛ/Volume perilymphₕᵤₘₐₙₛ

Formulations can be administered to the middle ear of a subject in need thereof, for example, by transtympanic injection. In some embodiments, a formulation is administered on or near the round window membrane via transtympanic injection. Formulations, in some embodiments, may also be administered on or near the round window or the crista fenestrae cochleae through entry via a post-auricular incision and surgical manipulation into or near the round window or the crista fenestrae cochleae area.

In some cases, administering can include using a syringe and small gauge needle, (e.g., 23 G to 30 G or smaller), wherein the needle is inserted through the tympanic membrane and guided to the area of the round window or crista fenestrae cochleae. The formulation is then deposited on or near the round window or crista fenestrae cochleae.

In some embodiments, a formulation can also be administered into the tympanic cavity or applied on the tympanic membrane or onto or in the cochlea by injection, direct instillation or perfusion of the inner ear compartments, or in surgical procedures including, cochleostomy, labyrinthotomy, mastoidectomy, stapedectomy, or endolymphatic sacculotomy.

The administering can include administering a therapeutically effective dose. In some embodiments, administering can include administering between about 5 and about 500 microliters (e.g., between about 5 82 L and about 400 82 L, between about 5 μL and about 300 μL, between about 5 82 L and about 200 82 L, between about 5 82 L and about 100 82 L, between about 5 μL and about 50 μL, between about 5 μL and about 25 μL, between about 5 μL and about 10 μL, between about 10 μL and about 500 μL, between about 25 μL and about 200 μL, between about 25 μL and about 500 μL, between about 50 μL and about 500 μL, between about 100 μL and about 500 μL, between about 200 μL and about 500 μL, between about 300 μL and about 500 μL, between about 400 μL and about 500 μL, between about 25 μL and about 300 μL, or between about 50 μL and about 200 4) of a formulation, such as any of the formulations provided herein. In some embodiments, administering can include administering about 50 μL, about 100 μL, or about 200 μL of a formulation, such as any of the formulations provided herein.

In some cases, before, during or after administration, a formulation effects a transition from a liquid state to a gel state. In some embodiments, the gel provides a therapeutically effective concentration of an active agent for a period of between about 5 days to about 6 months (e.g., about 5 days to about 1 week, about 5 days to about 2 weeks, about 5 days to about 3 weeks, about 5 days to about 1 month, about 5 days to about 2 months, about 5 days to about 3 months, about 5 days to about 4 months, about 5 days to about 5 months, about 1 week to about 6 months, about 2 weeks to about 6 months, about 3 weeks to about 6 months, about 1 month to about 6 months, about 2 months to about 6 months, about 3 months to about 6 months, about 4 months to about 6 months, about 5 months to about 6 months, about 2 weeks to about 2 months, or about 1 month to about 3 months) In some embodiments, the gel provides a therapeutically effective concentration of an active agent for at least 1 week (e.g., at least about 2 weeks, 3 weeks, 1 month, 2 months, 3 months, 4 months, 5 months, or 6 months).

Accordingly, provided herein are methods of treating a subject with an otic disease or condition. In some embodiments, provided herein are methods of treating a subject with an otic disease or condition including administering a therapeutically effective dose of a VEGF inhibitor. In some embodiments, provided herein are methods of treating a subject with an otic disease or condition including administering a therapeutically effective dose of a formulation as provided herein. In some embodiments, provided herein are methods of treating a subject with an otic disease or condition including administering a therapeutically effective dose of a VEGF inhibitor to an ear of a subject in need thereof. In some embodiments, provided herein are methods of treating a subject with an otic disease or condition including administering a therapeutically effective dose of a formulation as provided herein to an ear of a subject in need thereof. In some embodiments, provided herein are methods of treating a subject with an otic disease or condition including (i) identifying the subject as having an otic disease or condition, and (ii) administering a therapeutically effective dose of a VEGF inhibitor to an affected ear of the subject. In some embodiments, provided herein are methods of treating a subject with an otic disease or condition including (i) identifying the subject as having an otic disease or condition, and (ii) administering a therapeutically effective dose of a formulation as provided herein to an affected ear of the subject.

Identifying a subject with an otic disease or condition can be performed using any appropriate method. In some embodiments, identifying a subject with an otic disease or condition includes identifying one or more symptoms of an otic disease or condition in the subject, such as vertigo (e.g., having two episodes of vertigo, each lasting 20 minutes or longer but not longer than 12 hours), hearing loss (e.g., verified by a hearing test), tinnitus or a feeling of fullness in the ear, a combination thereof, or all thereof. In some embodiments, an otic disease or condition is identified when a subject exhibits one or more symptoms of an otic disease or condition and other known causes of the symptoms are excluded. In some embodiments, identifying a subject with an otic disease or condition includes identifying presence of BLB leakage (e.g., using contrast enhanced MRI). In some embodiments, after a subject is identified as having an otic disease or condition, the presence of BLB leakage is identified (e.g., using contrast enhanced MRI). Without being bound by any particular theory, it is believed that if a subject has BLB leakage, they are more likely to benefit from an anti-VEGF agent. For example, MRI can be used to detect hydrops and/or perilymphatic enhancement (which can often be considered to be a confirmatory sign of BLB leakage).

### Exemplary Embodiments

Embodiment 1 is a method of treating an otic disease or condition in a subject, the method comprising:
(i) identifying a subject as having the otic disease or condition; and
(ii) administering a therapeutically effective dose of a VEGF inhibitor to the subject.

Embodiment 2 is a method of treating an otic disease or condition in a subject, the method comprising:
administering a therapeutically effective dose of a VEGF inhibitor to a subject in need thereof.

Embodiment 3 is the method of embodiment 1 or embodiment 2, wherein the administering comprises systemic administration.

Embodiment 4 is the method of embodiment 1 or embodiment 2, wherein the administering comprises administering to an affected ear of the subject.

Embodiment 5 is the method of any one of embodiments 1, 2, or 4, wherein the VEGF inhibitor is administered in suspension, an ointment, a hydrogel, a liposome, a controlled release particle, an implantable device, or a combination thereof.

Embodiment 6 is the method of any one of embodiments 1, 2, or 4, wherein the VEGF inhibitor is administered in a formulation.

Embodiment 7 is a method of treating an otic disease or condition in a subject, the method comprising:
(i) preparing a formulation comprising a VEGF inhibitor; and
(ii) administering a therapeutically effective dose of the formulation to an affected ear of a subject in need thereof.

Embodiment 8 is a method of treating an otic disease or condition in a subject, the method comprising:
(i) identifying a subject as having the otic disease or condition;
(ii) preparing a formulation comprising a VEGF inhibitor; and
(iii) administering a therapeutically effective dose of the formulation to an affected ear of the subject.

Embodiment 9 is the method of embodiment 1 or embodiment 8, wherein identifying a subject as having the otic disease or condition comprises identifying one or more symptoms of an otic disease or condition in the subject.

Embodiment 10 is the method of embodiment 1 or embodiment 8, wherein identifying a subject as having the otic disease or condition includes identifying presence of the blood labyrinthine barrier (BLB) leakage.

Embodiment 11 is the method of any one of embodiments 1, 8, or 9, wherein following identifying the subject as having the otic disease or condition, the method further comprises identifying presence of BLB leakage.

Embodiment 12 is the method of any one of embodiments 6-11, wherein the administering comprises administering between about 5 μL and about 500 μL of the formulation.

Embodiment 13 is the method of any one of embodiments 6-11, wherein the administering comprises administering between about 25 μL, and about 200 μL of the formulation.

Embodiment 14 is the method of any one of embodiments 6-13, wherein the formulation comprises a hydrogel forming agent.

Embodiment 15 is the method of embodiment 14, wherein the formulation can form a hydrogel in the affected ear of the subject.

Embodiment 16 is the method of embodiment 14 or embodiment 15, wherein the hydrogel is selected from the group consisting of a temperature mediated phase transition gel, a shear thinning gel, an ionically crosslinked gel, a dendrimer gel, and a combination thereof.

Embodiment 17 is the method of any one of embodiments 14-16, wherein the hydrogel is a temperature mediated phase transition gel.

Embodiment 18 is the method of embodiment 17, wherein the hydrogel forming agent is a poloxamer. Embodiment 19 is the method of embodiment 18 wherein the hydrogel forming agent is poloxamer 407.

Embodiment 20 is the method of any one of embodiments 14-16, wherein the hydrogel is a shear thinning gel.

Embodiment 21 is the method of any one of embodiments 14-16, wherein the hydrogel is an ionically crosslinked gel.

Embodiment 22 is the method of embodiment 21, wherein the hydrogel forming agent is alginate.

Embodiment 23 is the method of any one of embodiments 14-16, wherein the hydrogel is a dendrimer gel.

Embodiment 24 is the method of any one of embodiments 14-23, wherein the hydrogel forming agent is present in the formulation in an amount of about 5% (w/w) to 30% (w/w).

Embodiment 25 is the method of embodiment 24, wherein the hydrogel forming agent is present in the formulation in an amount of about 15% (w/w) to 30% (w/w).

Embodiment 26 is the method of embodiment 24, wherein the hydrogel forming agent is present in the formulation in an amount of about 20% (w/w) to 30% (w/w).

Embodiment 27 is the method of any one of embodiments 6-26, wherein the VEGF inhibitor is present in the formulation in an amount of about 0.003% (w/w) to about 20% (w/w).

Embodiment 28 is the method of embodiment 27, wherein the VEGF inhibitor is present in the formulation in an amount of about 0.01% (w/w) to about 20% (w/w).

Embodiment 29 is the method of embodiment 27, wherein the VEGF inhibitor is present in the formulation in an amount of about 0.5% (w/w) to about 20% (w/w).

Embodiment 30 is the method of any one of embodiments 6-29, wherein the formulation further comprises one or more of an antimicrobial, an antioxidant, a buffer, a carrier, a chelator, a crystallization inhibitor, a detergent, a mucoadhesive agent, a penetration enhancer, a preservative, a solubilizing agent, a stabilizer, a tonicity agent, or a viscosity modifier.

Embodiment 31 is the method of any one of embodiments 6-30, wherein the formulation, following administration, provides sustained release of the VEGF inhibitor for at least 3 days.

Embodiment 32 is the method of any one of embodiments 6-30, wherein the formulation, following administration, provides sustained release of the VEGF inhibitor for at least 1 week.

Embodiment 33 is the method of any one of embodiments 6-30, wherein the formulation, following administration, provides sustained release of the VEGF inhibitor for at least 3 weeks.

Embodiment 34 is the method of any one of embodiments 6-30, wherein the formulation, following administration, provides sustained release of the VEGF inhibitor for at least 1 month.

Embodiment 35 is the method of any one of embodiments 6-30, wherein the formulation, following administration, provides sustained release of the VEGF inhibitor for at least 3 months.

Embodiment 36 is the method of any one of embodiments 4-35, wherein the administering of the VEGF inhibitor comprises injecting through the tympanic membrane.

Embodiment 37. The method of any one of embodiments 4-35, wherein the administering of the VEGF inhibitor comprises administering to the cochlea.

Embodiment 38 is the method of any one of embodiments 4-35, wherein the administering of the VEGF inhibitor comprises administration to the tympanic cavity.

Embodiment 39 is the method of any one of embodiments 4-35, wherein the administering comprises application of the VEGF inhibitor on the tympanic membrane.

Embodiment 40 is the method of any one of embodiments 4-35, wherein the administering comprises application of the VEGF inhibitor into the cochlea by injection, direct instillation or perfusion of the inner ear compartments.

Embodiment 41 is the method of any one of embodiments 4-35, wherein the administering occurs during a surgical procedure selected from the group consisting of cochleostomy, labyrinthotomy, mastoidectomy, stapedectomy, endolymphatic sacculotomy, and a combination thereof.

Embodiment 42 is the method of any one of embodiments 1-41, wherein the VEGF inhibitor is selected from the group consisting of altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof.

Embodiment 43 is the method of any one of embodiments 1-42, wherein the VEGF inhibitor comprises an antibody or antigen-binding fragment thereof.

Embodiment 44 is the method of embodiment 43, wherein the antibody or antigen-binding fragment thereof is selected from the group consisting of alacizumab, bevacizumab, icrucumab, ramucirumab, ranibizumab, and combinations thereof.

Embodiment 45 is the method of any one of embodiments 1-44, wherein the VEGF inhibitor comprises a decoy receptor.

Embodiment 46 is the method of embodiment 45, wherein the decoy receptor is aflibercept.

Embodiment 47 is the method of any one of embodiments 1-46, wherein the VEGF inhibitor comprises an allosteric modulator of a VEGFR.

Embodiment 48 is the method of embodiment 47, wherein the allosteric modulator of a VEGFR is cyclotraxin B.

Embodiment 49 is the method of any one of embodiments 1-48, wherein the VEGF inhibitor is at least 10-fold selective for VEGFR2 over another VEGFR.

Embodiment 50 is the method of any one of embodiments 1-49, wherein the VEGF inhibitor is at least 20-fold selective for VEGFR2 over another VEGFR.

Embodiment 51 is the method of any one of embodiments 1-49, wherein the VEGF inhibitor is at least 50-fold selective for VEGFR2 over another VEGFR.

Embodiment 52 is the method of any one of embodiments 1-51, wherein the amount of the VEGF inhibitor is sufficient to reduce edema and/or lymphatic dysfunction in an affected ear.

Embodiment 53 is the method of any one of embodiments 1-52, wherein the otic disease or condition is selected from the group consisting of Méniére's Disease (MD), Autoimmune Inner Ear Disease (AIED), sensorineural hearing loss (SNHL), noise-induced hearing loss (NIHL), age-related hearing loss, tinnitus, and a combination thereof.

Embodiment 54 is the method of any one of embodiments 1-52, wherein the otic disease or condition is selected from the group consisting of Autoimmune Inner Ear Disease (AIED), sensorineural hearing loss (SNHL), noise-induced hearing loss (NIHL), age-related hearing loss, tinnitus, and a combination thereof.

Embodiment 55 is the method of embodiment 53 or embodiment 54, wherein the SNHL is selected from the group consisting of sudden sensorineural hearing loss, sensorineural hearing loss associated with diabetes, and a combination thereof.

Embodiment 56 is the method of any one of embodiments 1-52, wherein the otic disease or condition is not Méniére's Disease.

Embodiment 57 is use of a VEGF inhibitor for the treatment of an otic disease or condition.

Embodiment 58 is use of a VEGF inhibitor in the manufacture of a medicament for the treatment of an otic disease or condition.

Embodiment 59 is the use of embodiment 57 or embodiment 58, wherein the VEGF inhibitor is selected from the group consisting of altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof.

Embodiment 60 is the use of any one of embodiments 57-59, wherein the VEGF inhibitor comprises an antibody or antigen-binding fragment thereof.

Embodiment 61 is the use of embodiment 60, wherein the antibody or antigen-binding fragment thereof is selected from the group consisting of alacizumab, bevacizumab, icrucumab, ramucirumab, ranibizumab, and combinations thereof.

Embodiment 62 is the use of any one of embodiments 57-61, wherein the VEGF inhibitor comprises a decoy receptor.

Embodiment 63 is the use of embodiment 62, wherein the decoy receptor is aflibercept.

Embodiment 64 is the use of any one of embodiments 57-63, wherein the VEGF inhibitor comprises an allosteric modulator of a VEGFR.

Embodiment 65 is the use of embodiment 64, wherein the allosteric modulator of a VEGFR is cyclotraxin B.

Embodiment 66 is the use of any one of embodiments 57-65, wherein the VEGF inhibitor is at least 10-fold selective for VEGFR2 over another VEGFR.

Embodiment 67 is the use of any one of embodiments 57-65, wherein the VEGF inhibitor is at least 20-fold selective for VEGFR2 over another VEGFR.

Embodiment 68 is the use of any one of embodiments 57-65, wherein the VEGF inhibitor is at least 50-fold selective for VEGFR2 over another VEGFR.

Embodiment 69 is the use of any one of embodiments 57-68, wherein the amount of the VEGF inhibitor is sufficient to reduce edema and/or lymphatic dysfunction in an affected ear.

Embodiment 70 is the use of any one of embodiments 57-69, where in the VEGF inhibitor is in the form of a suspension, an ointment, a hydrogels, a liposome, a controlled release particle, an implantable device, or a combination thereof.

Embodiment 71 is the use of any one of embodiments 57-70, wherein the VEGF inhibitor is part of a formulation.

Embodiment 72 is the use of embodiment 71, wherein an effective dose of the formulation is about 5 μL to about 500 μL of the formulation.

Embodiment 73 is the use of embodiment 71, wherein an effective dose of the formulation is about 50 μL to about 200 μL, of the formulation.

Embodiment 74 is the use of any one of embodiments 71-73, wherein the formulation comprises a hydrogel forming agent.

Embodiment 75 is the use of embodiment 74, wherein the formulation can form a hydrogel in the affected ear of the subject.

Embodiment 76 is the use of embodiment 74 or embodiment 75, wherein the hydrogel is selected from the group consisting of a temperature mediated phase transition gel, a shear thinning gel, an ionically crosslinked gel, a dendrimer gel, and a combination thereof.

Embodiment 77 is the use of any one of embodiments 74-76, wherein the hydrogel is a temperature mediated phase transition gel.

Embodiment 78 is the use of embodiment 77, wherein the hydrogel forming agent is a poloxamer.

Embodiment 79 is the use of embodiment 78 wherein the hydrogel forming agent is poloxamer 407.

Embodiment 80 is the use of any one of embodiments 74-76, wherein the hydrogel is a shear thinning gel.

Embodiment 81 is the use of any one of embodiments 74-76, wherein the hydrogel is an ionically crosslinked gel.

Embodiment 82 is the use of embodiment 81, wherein the hydrogel forming agent is alginate.

Embodiment 83 is the use of any one of embodiments 74-76, wherein the hydrogel is a dendrimer gel.

Embodiment 84 is the use of any one of embodiments 74-83, wherein the hydrogel forming agent is present in the formulation in an amount of about 5% (w/w) to 30% (w/w).

Embodiment 85 is the use of embodiment 84, wherein the hydrogel forming agent is present in the formulation in an amount of about 15% (w/w) to 30% (w/w).

Embodiment 86 is the use of embodiment 84, wherein the hydrogel forming agent is present in the formulation in an amount of about 20% (w/w) to 30% (w/w).

Embodiment 87 is the use of any one of embodiments 71-86, wherein the VEGF inhibitor is present in the formulation in an amount of about 0.003% (w/w) to about 20% (w/w).

Embodiment 88 is the use of embodiment 87, wherein the VEGF inhibitor is present in the formulation in an amount of about 0.01% (w/w) to about 10% (w/w).

Embodiment 89 is the use of embodiment 87, wherein the VEGF inhibitor is present in the formulation in an amount of about 0.5% (w/w) to about 10% (w/w).

Embodiment 90 is the use of any one of embodiments 71-89, wherein the formulation further comprises one or more of an antimicrobial, an antioxidant, a buffer, a carrier, a chelator, a crystallization inhibitor, a detergent, a mucoadhesive agent, a penetration enhancer, a preservative, a solubilizing agent, a stabilizer, a tonicity agent, or a viscosity modifier.

Embodiment 91 is the use of any one of embodiments 71-90, wherein the formulation, following administration, can provide sustained release of the VEGF inhibitor for at least 3 days.

Embodiment 92 is the use of any one of embodiments 71-91, wherein the formulation, following administration, can provide sustained release of the VEGF inhibitor for at least 1 week.

Embodiment 93 is the use of any one of embodiments 71-91, wherein the formulation, following administration, can provide sustained release of the VEGF inhibitor for at least 3 weeks.

Embodiment 94 is the use of any one of embodiments 71-91, wherein the formulation, following administration, can provide sustained release of the VEGF inhibitor for at least 1 month.

Embodiment 95 is the use of any one of embodiments 71-91 wherein the formulation, following administration, can provide sustained release of the VEGF inhibitor for at least 3 months.

Embodiment 96 is the use of any one of embodiments 57-95, wherein the otic disease or condition is selected from the group consisting of Méniére's Disease (MD), Autoimmune Inner Ear Disease (AIED), sensorineural hearing loss (SNHL), noise-induced hearing loss (NIHL), age-related hearing loss, tinnitus, and a combination thereof.

Embodiment 97 is the use of any one of embodiments 57-95, wherein the otic disease or condition is selected from the group consisting of Autoimmune Inner Ear Disease (AIED), sensorineural hearing loss (SNHL), noise-induced hearing loss (NIHL), age-related hearing loss, tinnitus, and a combination thereof.

Embodiment 98 is the use of embodiment 96 or embodiment 96, wherein the SNHL is selected from the group consisting of sudden sensorineural hearing loss, sensorineural hearing loss associated with diabetes, and a combination thereof.

Embodiment 99 is the use of any one of embodiments 57-95, wherein the otic disease or condition is not Méniére's Disease.

Embodiment 100 is a formulation for treatment of inner ear diseases and conditions comprising an effective amount of a vascular endothelial growth factor inhibitor or tyrosine kinase inhibitor to reduce edema and lymphatic dysfunction in an affected ear.

Embodiment 101 is the formulation of embodiment 100 comprising a compound which binds to VEGF or a VEGF receptor.

Embodiment 102 is the formulation of embodiment 101 wherein the compound is selected from the group consisting of antibodies, antibody fragments, and humanized antibodies, nucleic acids such as siRNA, miRNA, triple forming compounds, DNA and mRNA and molecules that express these inhibitors, low molecular weight and synthetic compounds.

Embodiment 103 is the formulation of embodiment 100 comprising a tyrosine kinase inhibitor with high affinity and specificity for VEGFR2.

Embodiment 104 is the formulation of embodiment 103 wherein the inhibitor is a small molecule inhibitor selected from the group of inhibitors inhibiting VEGF signaling consisting of pazopanib, sunitinib, sorafenib, axitinib, regorafenib, cabozantanib, lenvatinib, nintedanib, vandetanib and apatinib with high affinity and specificity for VEGFR2.

Embodiment 105 is the formulation of any of embodiments 100-104 further comprising therapeutic or prophylactic agents such as steroids, diuretics, vasodilators, antiinfectives and antihistamines.

Embodiment 106 is the formulation of any of embodiments 100-105 wherein the therapeutic agent is not a steroid.

Embodiment 107 is the formulation of any of embodiments 100-106 formulated for local, regional or systemic administration to an individual with MD to alleviate symptoms of the disease including edema and lymphatic dysfunction.

Embodiment 108 is the formulation of any of embodiments 100-107 wherein the agent is an effective amount of these compounds for delivery by intratympanic or intracochlear administration.

Embodiment 109 is the formulation of any of embodiments 100-108 for administration by topical, parenteral, subcutaneous, intraperitoneal or intranasal routes.

Embodiment 110 is the formulation of any of embodiments 100-109 for immediate release, sustained or controlled release.

Embodiment 111 is the formulation of any of embodiments 100-110 in the form of solutions, suspensions, ointments, hydrogels, liposomes, and controlled release particles.

Embodiment 112 is a method of alleviating edema and lymphatic dysfunction associated with inner ear disorders or conditions, including Méniére's Disease, comprising administering the formulation of any of embodiments 100-111 to an individual in need thereof.

Embodiment 113 is the method of embodiment 112 wherein the formulation is administered into the ear, through the tympanic membrane, or into the cochlear, by injection or surgical implant.

Embodiment 114 is the method of embodiment 112 wherein the formulation is administered systemically.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of treating an otic disease or condition in a subject, the method comprising:
(i) identifying a subject as having the otic disease or condition; and
(ii) administering a therapeutically effective dose of a VEGF inhibitor to the subject.

2. A method of treating an otic disease or condition in a subject, the method comprising:
administering a therapeutically effective dose of a VEGF inhibitor to a subject in need thereof.

3. The method of claim 1 or claim 2, wherein the administering comprises systemic administration.

4. The method of claim 1 or claim 2, wherein the administering comprises administering to an affected ear of the subject.

5. The method of any one of claim 1, 2, or 4, wherein the VEGF inhibitor is administered in suspension, an ointment, a hydrogel, a liposome, a controlled release particle, an implantable device, or a combination thereof.

6. The method of any one of claim 1, 2, or 4, wherein the VEGF inhibitor is administered in a formulation.

7. A method of treating an otic disease or condition in a subject, the method comprising:
(i) preparing a formulation comprising a VEGF inhibitor; and
(ii) administering a therapeutically effective dose of the formulation to an affected ear of a subject in need thereof.

8. A method of treating an otic disease or condition in a subject, the method comprising:
(i) identifying a subject as having the otic disease or condition;
(ii) preparing a formulation comprising a VEGF inhibitor; and
(iii) administering a therapeutically effective dose of the formulation to an affected ear of the subject.

9. The method of any one of claims 6-8, wherein the administering comprises administering between about 5 μL and about 500 μL of the formulation.

10. The method of any one of claims 6-8, wherein the administering comprises administering between about 25 μL and about 200 μL of the formulation.

11. The method of any one of claims 6-10, wherein the formulation comprises a hydrogel forming agent.

12. The method of claim 11, wherein the formulation can form a hydrogel in the affected ear of the subject.

13. The method of claim 11 or claim 12, wherein the hydrogel forming agent is present in the formulation in an amount of about 5% (w/w) to 30% (w/w).

14. The method of any one of claims 6-13, wherein the VEGF inhibitor is present in the formulation in an amount of about 0.003% (w/w) to about 20% (w/w).

15. The method of any one of claims 6-14, wherein the formulation, following administration, provides sustained release of the VEGF inhibitor for at least 3 days.

16. The method of any one of claims 6-14, wherein the formulation, following administration, provides sustained release of the VEGF inhibitor for at least 3 weeks.

17. The method of any one of claims 4-16, wherein the administering comprises injecting through the tympanic membrane.

18. The method of any one of claims 1-17, wherein the VEGF inhibitor is selected from the group consisting of altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof.

19. The method of any one of claims 1-18, wherein the VEGF inhibitor comprises an antibody or antigen-binding fragment thereof.

20. The method of claim 19, wherein the antibody or antigen-binding fragment thereof is selected from the group consisting of alacizumab, bevacizumab, icrucumab, ramucirumab, ranibizumab, and combinations thereof.

21. The method of any one of claims 1-20, wherein the VEGF inhibitor is at least 10-fold selective for VEGFR2 over another VEGFR.

22. The method of any one of claims 1-21, wherein the otic disease or condition is selected from the group consisting of Méniére's Disease (MD), Autoimmune Inner Ear Disease (AIED), sensorineural hearing loss (SNHL), noise-induced hearing loss (NIHL), age-related hearing loss, tinnitus, and a combination thereof.

23. Use of a VEGF inhibitor for the treatment of an otic disease or condition.

24. Use of a VEGF inhibitor in the manufacture of a medicament for the treatment of an otic disease or condition.

25. The use of claim 23 or claim 24, wherein the VEGF inhibitor is selected from the group consisting of altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof.

26. The use of any one of claims 23-25, wherein the VEGF inhibitor comprises an antibody or antigen-binding fragment thereof.

27. The use of claim 26, wherein the antibody or antigen-binding fragment thereof is selected from the group consisting of alacizumab, bevacizumab, icrucumab, ramucirumab, ranibizumab, and combinations thereof.

28. The use of any one of claims 23-27, wherein the VEGF inhibitor is at least 10-fold selective for VEGFR2 over another VEGFR.

29. The use of any one of claims 23-28, wherein the amount of the VEGF inhibitor is sufficient to reduce edema and/or lymphatic dysfunction in an affected ear.

30. The use of any one of claims 23-28, where in the VEGF inhibitor is in the form of a suspension, an ointment, a hydrogels, a liposome, a controlled release particle, an implantable device, or a combination thereof.

31. The use of any one of claims 22-29, wherein the VEGF inhibitor is part of a formulation.

32. The use of claim 31, wherein the formulation comprises a hydrogel forming agent.

33. The use of claim 32, wherein the formulation can form a hydrogel in the affected ear of the subject.

34. The use of claim 32 or claim 33, wherein the hydrogel forming agent is present in the formulation in an amount of about 5% (w/w) to 30% (w/w).

35. The use of any one of claims 31-34, wherein the VEGF inhibitor is present in the formulation in an amount of about 0.003% (w/w) to about 20% (w/w).

36. The use of any one of claims 31-35, wherein the formulation, following administration, can provide sustained release of the VEGF inhibitor for at least 3 days.

37. The use of any one of claims 31-36, wherein the formulation, following administration, can provide sustained release of the VEGF inhibitor for at least 3 weeks.

38. The use of any one of claims 31-37, wherein the otic disease or condition is selected from the group consisting of Méniére's Disease (MD), Autoimmune Inner Ear Disease (AIED), sensorineural hearing loss (SNHL), noise-induced hearing loss (NIHL), age-related hearing loss, tinnitus, and a combination thereof.


SEQUENCE LISTING (8 sequences)

SEQ ID NO: 1 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 2 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 3 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 4 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 5 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 6 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 7 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 8 - protein, 221 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR